SEQUENCE LISTING (27 sequences)

SEQ ID NO: 1 - DNA, 18 bp, Artificial
  F1 Forward Primer
tctwggwggmatgggttg

SEQ ID NO: 2 - protein, 6 aa, Artificial
  Coded by F1 Forward Primer
LGGMGC

SEQ ID NO: 3 - DNA, 18 bp, Artificial
  R1 Reverse Primer
trtaygcyarctcrtacc

SEQ ID NO: 4 - protein, 6 aa, Artificial
  Coded by R1 Reverse Primer
WYELAY

SEQ ID NO: 5 - DNA, 20 bp, Artificial
  P Forward Primer
accatgtcaggaacaaaagc

SEQ ID NO: 6 - DNA, 23 bp, Artificial
  PR Reverse Primer
ttaatttaatggaacctcaaccg

SEQ ID NO: 7 - DNA, 32 bp, Artificial
  F2 Forward Primer
tcgaggatgtcgcttcaccgatttggaaacac

SEQ ID NO: 8 - DNA, 33 bp, Artificial
  R2 Reverse Primer
gtttccaaatcggtgaagcgacatcctcgatgg

SEQ ID NO: 9 - DNA, 25 bp, Artificial
  F3 Forward Primer
taggatccatgtcaggaacaaaagc

SEQ ID NO: 10 - DNA, 30 bp, Artificial
  R3 Reverse Primer
tagagctcttaatttaatggaacctcaacc

SEQ ID NO: 11 - DNA, 30 bp, Artificial
  R4 Reverse Primer
taggatccttaatttaatggaacctcaacc

SEQ ID NO: 12 - DNA, 17 bp, Artificial
  F4 Forward Primer
atgtcaggaacaaaagc

SEQ ID NO: 13 - DNA, 22 bp, Artificial
  R5 Reverse Primer
taatttaatggaacctcaaccg

SEQ ID NO: 14 - DNA, 24 bp, Artificial
  F5 Forward Primer
gcaatgacgtccattaacgtaaag

SEQ ID NO: 15 - DNA, 21 bp, Artificial
  R6 Reverse Primer
ttaggaccgaccgttttgggc

SEQ ID NO: 16 - DNA, 29 bp, Artificial
  F6 Forward Primer
tatctagaatgacgtccattaacgtaaag

SEQ ID NO: 17 - DNA, 27 bp, Artificial
  R7 Reverse Primer
atggtaccttaggaccgaccgttttgg

SEQ ID NO: 18 - DNA, 22 bp, Artificial
  NN-3 Primer
tttcttcgccacttgtcactcc

SEQ ID NO: 19 - DNA, 21 bp, Artificial
  NN-4 Primer
cgcgctatattttgttttcta

SEQ ID NO: 20 - DNA, 32 bp, Artificial
  OM087 Primer
agagagagggatccatgagtgtgataggtagg

SEQ ID NO: 21 - DNA, 33 bp, Artificial
  OM088 Primer
gaggaagaaggatccgggtctatatactactct

SEQ ID NO: 22 - protein, 503 aa, Tropaeolum majus
MSGTKATSVSVPLPDFKQSVNLKYVKLGYHYSITHAMYLFLTPLLLIMSAQISTFSIQDFHHLYNHLILHNLSSLILCIALLLFVLTLYFLTRPTPVYLLNFSCYKPDAIHKCDRRRFMDTIRGMGTYTEENIEFQRKVLERSGIGESSYLPPTVFKIPPRVYDAEERAEAEMLMFGAVDGLFEKISVKPNQIGVLVVNCGLFNPIPSLSSMIVNRYKMRGNVFSYNLGGMGCSAGVISIDLAKDLLQVRPNSYALVVSLECISKNLYLGEQRSMLVSNCLFRMGGAAILLSNKMSDRWRSKYRLVHTVRTHKGTEDNCFSCVTRKEDSDGKIGISLSKNLMAVAGDALKTNITTLGPLVLPMSEQLLFFATLVGKKVFKMKLQPYIPDFKLAFEHFCIHAGGRAVLDELEKNLKLSSWHMEPSRMSLYRFGNTSSSSLWYELAYSEAKGRIKKGDRVWQIAFGSGFKCNSAVWKALRNVNPAEEKNPWMDEIHLFPVEVPLN

SEQ ID NO: 23 - DNA, 1765 bp, Tropaeolum majus
agttttttttgttgagaataaccatgtcaggaacaaaagcaacatcagtttctgttccactgcctgatttcaagcaatcagttaatctaaaatatgttaaacttggttatcattactcgatcactcatgcaatgtatctttttctaacccctcttcttctcataatgtctgctcaaatctcaactttctctattcaagattttcaccatctttataaccatcttatcctccacaatctctcatcccttatcctatgcatcgctctcctcctcttcgtcttaaccctctatttccttactcgtcccacgcctgtttatttactcaacttctcttgttacaaaccggatgctattcacaaatgcgaccgccgtcgtttcatggacaccattcgtggaatgggtacttatacggaagagaacatcgagtttcaaaggaaagttctagaaaggtccggaataggggaatcgtcttatcttcctccgactgtgtttaaaattcctcctagggtttacgatgcggaggaacgcgcggaggctgagatgctgatgttcggtgcggttgatgggcttttcgagaaaatatctgttaaaccgaatcaaatcggggttttggttgtgaattgtgggttgtttaatccgataccgtctttatcttccatgattgtgaatcgctacaagatgagagggaatgtttttagttataatttgggtggaatgggttgtagtgcgggtgtgatttcgattgatcttgctaaagatcttcttcaggttcgtcccaactcatatgctttggtggttagtttggaatgtatctcgaagaacttgtatctcggtgaacaaagatcgatgcttgtttccaactgtttgtttcgaatgggtggggcggcgattttgctttcgaataaaatgtcggatcgatggagatcaaagtatagattggttcatacggttcgaacccacaagggtaccgaggataactgcttttcttgcgtaactagaaaggaagactcggacgggaagatcggtatttctttatcgaagaacctaatggctgttgccggagacgcattgaagactaatatcacaaccctcggaccacttgttctacccatgtcggaacaattactcttcttcgctactttggtcggaaaaaaggttttcaagatgaagctacagccgtatataccggatttcaagttggctttcgagcatttctgtattcatgcaggtggaagagctgttctggatgaattggagaagaacttgaagctttcgagttggcatatggaaccatcgaggatgtcgctttaccgatttggaaacacgtcgagtagttcgctttggtacgagttggcttattcggaggcgaaagggagaataaagaagggagatcgagtatggcaaatcgcgtttgggtcgggatttaagtgtaacagtgcggtgtggaaggctctaaggaatgttaatccggcggaagagaaaaatccttggatggatgagattcacctatttccggttgaggttccattaaattaaaacctatcttcaagttacaagttgttgttgttgtttcattaggtttaataataagctaatatggaaagcctttctactctcttttttttccacttttttttttcaatttcagagttgggtcttagttgtatcatctacatgagtgtattcgctatgcgctattcgctattcgctattcactagttaataaaatcaaacgtccaaaaaaaaaaaaaaaaaaaaaaaaaaaa

SEQ ID NO: 24 - protein, 506 aa, Crambe abyssinica
MTSINVKLLYHYVITNLFNLCFFPLTAIVAGKASRLTIDDLHHLYYSYLQHNVITIAPLFAFTVFGSILYIVTRPKPVYLVEYSCYLPPTQCRSSISKVMDIFYQVRKADPFRNGTCDDSSWLDFLRKIQERSGLGDETHGPEGLLQVPPRKTFAAAREETEQVIVGALKNLFENTKVNPKDIGILVVNSSMFNPTPSLSAMVVNTFKLRSNVRSFNLGGMGCSAGVIAIDLAKDLLHVHKNTYALVVSTENITYNIYAGDNRSMMVSNCLFRVGGAAILLSNKPRDRRRSKYELVHTVRTHTGADDKSFRCVQQGDDENGKTGVSLSKDITEVAGRTVKKNIATLGPLILPLSEKLLFFVTFMAKKLFKDKVKHYYVPDFKLAIDHFCIHAGGRAVIDVLEKNLGLAPIDVEASRSTLHRFGNTSSSSIWYELAYIEAKGRMKKGNKVWQIALGSGFKCNSAVWVALSNVKASTNSPWEHCIDRYPVKIDSDSAKSETRAQNGRS

SEQ ID NO: 25 - DNA, 1521 bp, Crambe abyssinica
atgacgtccattaacgtaaagctcctttaccattacgtcataaccaacctttttaacctctgtttctttccgttaacggcgatcgtcgccgggaaagcctctcggcttaccatagacgatcttcaccacttatattattcctatctccaacacaacgtcataaccatagctccactctttgcctttaccgttttcggttcgattctctacatcgtgacccggcccaaaccggtttacctcgttgagtactcatgctaccttccaccaacgcagtgtagatcaagtatctccaaggtcatggatatattttatcaagtaagaaaagctgatccttttcgtaacgggacatgcgatgactcgtcctggcttgacttcttgaggaagattcaagaacgttcaggtctaggcgacgaaactcacggccccgagggactgcttcaggtccctccccggaagacttttgcggcggcgcgtgaagagacggagcaagtaatcgtcggtgcgctgaaaaatctattcgagaacaccaaagttaaccctaaagatataggtatacttgtggtgaactcaagcatgtttaatccaactccttcactctcagcgatggtcgttaatactttcaagctccgaagtaacgtaagaagctttaaccttggtggcatgggttgtagtgctggcgttatagccattgatctggctaaggacttgttgcatgtccataaaaacacgtatgctcttgtggtgagcacagagaacatcacttataacatttacgctggcgataatagatccatgatggtttcaaactgcttgttccgtgttggcggggccgctattttgctctccaacaagcctagagatcgaagacggtccaaatacgagctagttcacacggtccgaacacataccggagctgatgacaagtctttccgatgcgtccaacaaggagacgatgagaacggcaaaaccggagtgagtttgtccaaggacataaccgaggttgctggtcgaacggttaagaaaaacatagcaacattgggtcctttgattcttcctttaagcgagaaacttctttttttcgttaccttcatggccaagaaacttttcaaagataaagttaagcattactatgtcccggacttcaagcttgctattgaccatttttgtatacatgcgggaggcagagccgtgatcgatgtgctagagaagaatttaggcctagcaccgatcgatgtagaggcatcaagatcaacgttacatagatttggtaacacatcatctagctcaatatggtatgagttggcatacatagaggcaaaaggaaggatgaagaaaggtaataaagtttggcagattgctttagggtcaggctttaagtgtaacagtgcggtttgggtagctttaagcaatgtcaaggcttcgacaaatagtccttgggaacattgcatcgatagatacccggttaaaattgattctgattcagctaagtcagagactcgtgcccaaaacggtcggtcctaa

SEQ ID NO: 26 - protein, 506 aa, Arabidopsis sp.
MTSVNVKLLYRYVLTNFFNLCLFPLTAFLAGKASRLTINDLHNFLSYLQHNLITVTLLFAFTVFGLVLYIVTRPNPVYLVDYSCYLPPPHLKVSVSKVMDIFYQIRKADTSSRNVACDDPSSLDFLRKIQERSGLGDETYSPEGLIHVPPRKTFAASREETEKVIIGALENLFENTKVNPREIGILVVNSSMFNPTPSLSAMVVNTFKLRSNIKSFNLGGMGCSAGVIAIDLAKDLLHVHKNTYALVVSTENITQGIYAGENRSMMVSNCLFRVGGAAILLSNKSGDRRRSKYKLVHTVRTHTGADDKSFRCVQQEDDESGKIGVCLSKDITNVAGTTLTKNIATLGPLILPLSEKFLFFATFVAKKLLKDKIKHYYVPDFKLAVDHFCIHAGGRAVIDELEKNLGLSPIDVEASRSTLHRFGNTSSSSIWYELAYIEAKGRMKKGNKAWQIALGSGFKCNSAVWVALRNVKASANSPWQHCIDRYPVKIDSDLSKSKTHVQNGRS

SEQ ID NO: 27 - DNA, 1521 bp, Arabidopsis sp.
atgacgtccgttaacgttaagctcctttaccgttacgtcttaaccaactttttcaacctctgtttgttcccgttaacggcgttcctcgccggaaaagcctctcggcttaccataaacgatctccacaacttcctttcctatctccaacacaaccttataacagtaactttactctttgctttcactgttttcggtttggttctctacatcgtaacccgacccaatccggtttatctcgttgactactcgtgttaccttccaccaccgcatctcaaagttagtgtctctaaagtcatggatattttctaccaaataagaaaagctgatacttcttcacggaacgtggcatgtgatgatccgtcctcgctcgatttcctgaggaagattcaagagcgttcaggtctaggtgatgagacgtacagtcctgagggactcattcacgtaccaccgcggaagacttttgcagcgtcacgtgaagagacagagaaggttatcatcggtgcgctcgaaaatctattcgagaacaccaaagttaaccctagagagattggtatacttgtggtgaactcaagcatgtttaatccaactccttcgctatccgctatggtcgttaatactttcaagctccgaagcaacatcaaaagctttaatctaggaggaatgggttgtagtgctggtgttattgccattgatttggctaaagacttgttgcatgttcataaaaacacttatgctcttgtggtgagcactgagaacatcacacaaggcatttatgctggagaaaatagatcaatgatggttagcaattgcttgtttcgtgttggtggggccgcgattttgctctctaacaagtcgggagaccggagacggtccaagtacaagctagttcacacggtccgaacgcatactggagctgatgacaagtcttttcgatgtgtgcaacaagaagacgatgagagcggcaaaatcggagtttgtctgtcaaaggacataaccaatgttgcggggacaacacttacgaaaaatatagcaacattgggtccgttgattcttcctttaagcgaaaagtttctttttttcgctaccttcgtcgccaagaaacttctaaaggataaaatcaagcattactatgttccggatttcaagcttgctgttgaccatttctgtattcatgccggaggcagagccgtgatcgatgagctagagaagaacttaggactatcgccgatcgatgtggaggcatctagatcaacgttacatagatttgggaatacttcatctagctcaatttggtatgaattagcatacatagaggcaaagggaagaatgaagaaagggaataaagcttggcagattgctttaggatcagggtttaagtgtaatagtgcggtttgggtggctctacgcaatgtcaaggcatcggcaaatagtccttggcaacattgcatcgatagatatccggttaaaattgattctgatttgtcaaagtcaaagactcatgtccaaaacggtcggtcctaa

(19) United States
(12) Patent Application Publication
(10) Pub. No.: US 2007/0204370 A1
(43) Pub. Date: Aug. 30, 2007

(54) Fatty Acid Elongase (Fae) Genes And Their Utility In Increasing Erucic Acid And Other Very Long-Chain Fatty Acid Proportions In Seed Oil.

(75) Inventors: Elzbieta Mietkiewska, Saskatoon (CA); David C. Taylor, Saskatoon (CA); Vesna Katavic, Saskatoon (CA)

Correspondence Address: BORDEN LADNER GERVAIS LLP, WORLD EXCHANGE PLAZA, 100 QUEEN STREET SUITE 1100, OTTAWA, ON, K1P 1J9, CA

(73) Assignee: NATIONAL RESEARCH COUNCIL OF CANDA, Ottawa, ON (CA)

(21) Appl. No.: 10/596,024

(22) PCT Filed: Nov. 24, 2004
(86) PCT No.: PCT/CA04/02021

§ 371 Date: May 15, 2007

Related U.S. Application Data
(60) Provisional application No. 60/524,645, filed on Nov. 25, 2003.

(51) Int. Cl. A01H 5/00; C12N 1/00; C12N 15/82; C12N 5/04

(52) U.S. Cl. 800/281; 800/306; 435/468; 435/419; 435/254.2

(57) ABSTRACT

This invention relates to seeds of plant, plants themselves and cells of such plants which comprise a heterologous gene coding for a plant (such as nasturtium (Tropaeolum majus) or Crambe abyssinica) fatty acid elongase (FAE) gene or allelic variant thereof, or combinations of one or both of these FAE genes with an Arabidopsis fatty acid elongase 1 (FAE1) gene, in co-transformation, in reading frame alignment with a promoter capable of increasing expression of said gene(s), when said transformed plant cell is in a seed, said plant cell or seed being capable of producing an increase in proportion of a very long chain monounsaturated or saturated fatty acids when compared with the proportions of said fatty acids in a control plant cell or seed lacking said heterologous FAE gene or genes. The invention also relates to combinations of these fatty acid elongase genes by traditional crossing, sufficient to increase the proportion of very long chain monounsaturated or saturated fatty acids in the seed oil of the progeny compared to the proportion of said fatty acids in either of the parental lines.

### BACKGROUND TO THE INVENTION

Very long chain fatty acids (VLCFAs) with 20 carbons or more are widely distributed in nature. In plants they are mainly found in epicuticular waxes and in the seed oils of a number of plant species, including members of the Brassicaceae, Limnantheceae, Simmondsia and Tropaeolaceae. A strategic goal in oilseed modification is to genetically manipulate high erucic acid (HEA) germplasm of the Brassicaceae to increase the content of erucic acid (22:1 Δ13) and other strategic VLCFAs in the seed oil for industrial niche market needs. Erucic acid and its derivatives are feedstocks in manufacturing slip-promoting agents, surfactants, plasticizers, nylon 1313, and surface coatings and more than 1000 patents have been issued. The current market for high erucate oils exceeds $120 million U.S./annum. Worldwide erucic acid demand is predicted to increase from about 40 million pounds (M pds) in 1990 to about 80 M pds by the year 2010. Similarly, demand for the derivative, behenic acid, is predicted to triple to about 102 M pds by 2010. In recent years, production has increased to meet market needs and high erucic acreage in western Canada is currently at a record high. A Brassica cultivar containing erucic acid levels approaching 80% would significantly reduce the cost of producing erucic acid and its derivatives and could meet the forecast demand for erucic and behenic acids as renewable, environmentally-friendly industrial feedstocks.

VLCFAs are synthesized outside the plastid by a membrane bound fatty acid elongation complex (elongase) using acyl-CoA substrates. The first reaction of elongation involves condensation of malonyl-CoA with a long chain substrate producing a 3-ketoacyl-CoA. Subsequent reactions are reduction of 3-hydroxyacyl-CoA, dehydration to an enoyl-CoA, followed by a second reduction to form the elongated acyl-CoA. The 3-ketoacyl-CoA synthase (KCS) catalyzing the condensation reaction plays a key role in determining the chain length of fatty acid products found in seed oils and is the rate-limiting enzyme for seed VLCFA production. Hereafter the terms elongase and FAE will signify 3-ketoacyl-CoA synthase condensing enzyme genes/proteins. The composition of the fatty acyl-CoA pool available for elongation and the presence and size of the neutral lipid sink are additional important factors influencing the types and levels of VLCFAs made in particular cells.

Our knowledge of the mechanism of elongation and properties of FAE1 and other elongase condensing enzymes is, in part, limited by their membrane-bound nature: as such they are more difficult to isolate and characterize than soluble condensing enzymes. The genes encoding FAE1 and its homologs have been cloned from Arabidopsis thaliana and from Brassica napus (two homologous sequences, Bn-FAE1.1 and Bn-FAE 1.2).

Site-directed mutagenesis experiments have been carried out on the Arabidopsis FAE1 to decipher the importance of cysteine and histidine as residues in condensing enzyme catalysis. Results have shown that cysteine²²³ and four histidine residues are essential for the enzyme activity.

In this work, we selected Tropaeolum majus, garden nasturtium, as a source of the elongase involved in VLCFA synthesis based on the fact that this plant is capable of producing significant amounts of erucic acid (70-75% of total fatty acid) and accumulates trierucin as the predominant triacylglycerol (TAG) in its seed oil. Here, we report the isolation of a nasturtium FAE gene and demonstrate the involvement of its encoded protein in the elongation of saturated and especially monounsaturated fatty acids. We also selected Crambe abyssinica as a second source of an elongase gene since Crambe is grown, particularly in the US, as an alternative crop for high erucic acid 46-50% (wt/wt) oil.

This invention relates to a nasturtium cDNA encoding an “elongase” (condensing enzyme) with a high specificity for eicosenoyl moieties which can be utilized to engineer seed oil crops for production of high erucic acid oils. This invention also relates to a Crambe cDNA encoding an elongase with a strong capability to synthesize erucic acid.

There is interest in modifying the seed oil fatty acid composition and content of oilseeds by molecular genetic means to provide a dependable source of Super High Erucic Acid Rapeseed (SHEAR) oil for use as an industrial feedstock.

Nonetheless, to date, increases in the content of some strategic fatty acids have been achieved by introduction of various fatty acid biosynthesis genes in oilseeds. Some examples include:
Expression of a medium chain fatty acid thioesterase from California Bay, in Brassicaceae to increase the lauric acid content. (Calgene)
Expression of an anti-sense construct to the Δ9 desaturase in Brassicaceae to increase the stearic acid content. (Calgene)
Increased proportions of oleic acid by co-suppression using constructs encoding plant microsomal desaturases. (DuPont/Cargill)
Expression of a Jojoba “elongase” 3-keto-acyl-CoA synthase in low erucic acid (canola) B. napus cultivars to increase the level of erucic acid; the effect following expression in high erucic acid cultivars was negligible (Calgene).

However, there has not been an elongase gene identified or characterized as encoding an FAE with the ability to produce 22:1 beyond the level already existing in HEAR B. napus cultivars.

We considered that the isolated FAE “elongase” homolog from Tropaeolum majus (garden nasturtium) with GenBank Accession No. AY082610 (published on Mar. 6, 2002), could be used to engineer plants to produce seed oils highly enriched in erucic acid. We found that to date, this is the first “elongase” transgene experiment to result in an 8-fold increase in the proportions erucic acid in Arabidopsis plants. When expressed in B. carinata, the nasturtium FAE gene resulted in an increase in erucic acid proportions of up to 6%. When co-expressed with the Arabidopsis FAE gene in B. carinata the result was an increase in erucic acid proportions by up to 16-18%.

We also cloned and functionally expressed in yeast, a Crambe FAE gene (GenBank Accession No. AY793549), resulting in the accumulation of 20:1 c 11 and 22:1 c13, fatty acids not found in wild-type yeast control lines.

To our knowledge the nearest art relates to an elongase gene (FAE1) from Arabidopsis which was cloned and published as: James, D. W. Jr., Lim, E., Keller, J., Plooy, I., Ralston, E. and Dooner, H. K. (1995) Directed tagging of the Arabidopsis FATTY ACID ELONGATION1 (FAE1) gene with the maize transposon activator. The Plant Cell 7: 309-319 (1995).

The reader is also referred to sequences 7, 9, 11, 13, 15, 17, 19, 21, 23, 25, 27, 29, 35, 37, 39, 41 from Jaworski, J. G. and Blacklock, B. J. Patent Application WO0194565 as well as sequences 19, 20, 21, 22, 23 from Kunst and Clemens, Regulation of embryonic transcription in plants. Patent Application WO0111061; 15 Feb. 2001; University of British Columbia (CA).

### SUMMARY OF THE INVENTION

The invention relates to an expression vector for transforming a cell, the expression vector comprising a gene coding for a plant fatty acid elongase in reading frame alignment with a promoter capable of increasing expression of the gene, when the transformed cell is in a seed, sufficient to increase the proportion of very long chain monounsaturated fatty acid when compared with a control cell. The expression vector may, for example, comprise a gene encoding a nasturtium (Tropaeolum majus) fatty acid elongase gene, a Crambe fatty acid elongase gene or an Arabidopsis fatty acid elongase 1 (FAE1) gene. The expression vector may further, for example, comprise a gene encoding a nasturtium (Tropaeolum majus) fatty acid elongase gene or a Crambe fatty acid elongase gene, or combinations of one or both of these FAE genes with an Arabidopsis fatty acid elongase 1 (FAE1) gene in co-transformation experiments in reading frame alignment with a promoter capable of increasing expression of said gene(s), when said transformed cell is in a seed, sufficient to increase in proportion of very long chain monounsaturated fatty acid when compared with a control cell. The invention also relates to cell comprising a heterologous gene coding for a heterologous plant fatty acid elongase or allelic variant thereof, said cell being capable of producing an increase in proportion of a very long chain monounsaturated fatty acid when compared a control cell lacking said heterologous gene. The cell may, for example, be a fungal, yeast or plant cell, especially a plant seed cell. The cell may, for example, comprise a heterologous gene coding for a nasturtium, Crambe, or Arabidopsis fatty acid elongase gene or allelic variant thereof, said cell being capable of producing an increase, preferably at least a 10% increase, in proportion of a very long chain monounsaturated fatty acid (e.g. erucic acid) when compared with a control cell lacking said heterologous gene. The increase can be larger, e.g. up to about eight-fold. In a plant cell of the invention the heterologous gene may code for a 3-ketoacyl-CoA synthase. The plant cell of the invention may additionally comprise a further heterologous gene coding for an additional heterologous plant fatty acid elongase or allelic variant thereof or a heterologous plant desaturase gene or allelic variant thereof. The plant cell of the invention preferably is capable of producing oil with an increased content of erucic acid or other very long chain fatty acid (C₂₀ or greater). The invention also relates to seeds and plants comprising such cells and the use of such vectors to produce such cells, seeds and plants. The plant preferably is a dicotyledon, especially a member of the Brassicaceae, Limnanthaceae, Tropaeolaceae or Simmondsia. Plants of the genus Brassica and Linum usitatissimu L. are especially preferred.

The invention also relates to a method for altering erucic acid content of a plant-derived oil which method comprises cultivating a plant of the invention and then extracting a plant-derived oil therefrom which oil has altered erucic acid content. Use of a heterologous plant fatty acid elongase gene for altering erucic acid content in a plant is also contemplated. Use of a heterologous plant fatty acid elongase gene for altering the very long chain fatty acid content (C₂₀ or greater) in a plant is further contemplated.

The fatty acid elongase (often designated FAE or 3-ketoacyl-CoA synthase (KCS)) is a condensing enzyme and is the first component of the elongation complex involved in synthesis of erucic acid (22:1) in seeds of Tropaeolum majus (garden nasturtium). Using a degenerate primers approach, a cDNA of a putative embryo FAE was obtained showing high homology to known plant elongases. This cDNA contains a 1512-nucleotide open reading frame (ORF) that encodes a protein of 504 amino acids. A genomic clone of the nasturtium FAE was isolated and sequence analyses indicated the absence of introns. Northern hybridization showed the expression of this nasturtium FAE gene to be restricted to the embryo. Southern hybridization revealed the nasturtium 3-ketoacyl-CoA synthase to be encoded by a small multigene family. To establish the function of the elongase homolog, the cDNA was introduced into two different heterologous chromosomal backgrounds, Arabidopsis (A. thaliana) and tobacco (Nicotiana tabacum), under the control of a seed-specific (napin) promoter and the tandem 35S promoter, respectively. Seed-specific expression resulted in up to an 8-fold increase in erucic acid proportions in Arabidopsis seed oil. Constitutive expression in transgenic tobacco tissue resulted in increased proportions of very long chain saturated fatty acids. These results indicate that the nasturtium FAE gene encodes a condensing enzyme involved in the biosynthesis of very-long-chain fatty acids, utilizing monounsaturated and saturated acyl substrates. It shows utility for directing or engineering increased synthesis of erucic acid in other plants. Using a PCR based approach, a cDNA of an embryo-specific FAE was cloned from Crambe abyssinica.

### BRIEF DESCRIPTION OF THE FIGURES

FIG. 1. Substrate specificity of elongase(s) from mid-developing nasturtium (T. majus) embryos. 200 μg of protein from a 15,000×g particulate fraction was used in the elongase assay. Reaction conditions were as described in the Detailed Description of the Invention. Results represent the average of three replicates. For each [1-¹⁴C]-acyl-CoA substrate, the relative proportional distribution of radiolabeled fatty acid elongation product(s) is (are) demarcated.

FIG. 2 A: Comparison of the amino acid sequences of the nasturtium FAE homolog (NasFAE; accession no. AY0826190) with fatty acid elongase 1 (FAE1) and related 3-ketoacyl-CoA synthases from other plant species. The alignment contains the sequences of the corn (ZeaFAE), Limnanthes (LimFAE), jojoba (SimFAE), Arabidopis (AraFAE) Brassica (BraFAE) and two Arabidopis 3-ketoacyl-CoA synthases associated with wax synthesis (AraKCS, AraCUT). The GenBank Accession numbers for the sequences shown are AJ292770 (ZeaFAE), AF247134 (LimFAE), U37088 (SimFAE), AF053345 (AraKCS), AF129511 (AraACUT), U29142 (AraFAE), AF009563 (BraFAE). Conserved cysteine and histidine residues are labeled with diamonds and triangles, respectively. Tyrosine at position 429 in the nasturtium FAE polypeptide is indicated by an asterisk. B: Dendrogram of the 3-ketoacyl-CoA synthase gene family based on the amino acid sequences. The alignment was carried out by the Clustal W method using Lasergene analysis software (DNAStar, Madison, Wis.) GenBank accession numbers: AF247134 (LimFAE), U37088 (SimFAE), AY082610 (NasFAE), AJ292770 (ZeaFAE), AF053345 (AraKCS), U29142 (AraFAE), AF009563 (BraFAE), AF129511 (AraCUT).

FIG. 3. Hydropathy analysis of T. majus FAE. A: Hydropathy plot of FAE indicating the presence of several hydrophobic regions. B: Schematic representation of the putative transmembrane domains of T. majus FAE amino-acid sequence as predicted by TMAP analysis (Persson and Argos 1994). Numbers shown in the boxes correspond to the residues of each domain in FAE.

FIG. 4. Northern and Southern analyses of T. majus FAE.

A: Northern analysis of FAE gene expression in T. majus. Total RNA was isolated from roots (RT), leaves (LF), petals (PL) and embryos (EO). B: Southern blot analysis of the FAE gene in T. majus. Genomic DNA was digested with restriction enzymes: EcoRI (lane 1), AccI (lane 2), NcoI (lane 3) and HindIII (lane 4).

FIG. 5. A. Proportions of 20:1 Δ11 and 22:1 Δ13 in seed oils from non-transformed A. thaliana ecotype Wassilewskija (WS-Con), two plasmid only transgenic control lines (RD1- and RD-15), and the eighteen best A. thaliana T₂ transgenic lines expressing the T. majus FAE gene under control of the napin promoter. B. Proportions of 18:0, 20:0, 22:0 and 24:0 in seed oils from non-transformed A. thaliana ecotype Wassilewskija (WS-Con), two plasmid only transgenic control lines (RD1- and RD-15), and the eighteen best A. thaliana T₂ transgenic lines expressing the T. majus FAE gene under control of the napin promoter. The values are the average±SD of three determinations performed on 200-seed lots.

FIG. 6. The accumulation of erucic acid (22:1) in T₁ mature seeds of nor-transformed Brassica carinata wild-type control (ntB, Black bar) and Brassica carinata transformed with the nasturtium FAE gene (NF Lines, Gray bars).

FIG. 7. The accumulation of erucic acid (22:1) in T₁ mature seeds of non-transformed Brassica carinata wild type control (ntB, Black bar) and Brassica carinata transgenic lines carrying both the Arabidopsis FAE1 and nasturtium FAE genes (Lines 6A through 33G; Gray bars).

FIG. 8. The accumulation of 20:1 Δ5 and 22:2 Δ5, Δ13 in T₂ mature seeds of Arabidopsis thaliana-ecotype Wassilewskija non-transformed wild-type (nt-WT) and empty vector only controls, and Arabidopsis thaliana of the same ecotype co-transformed with the nasturtium FAE and Limnanthes D5 desaturase genes (NFPC/D5 Lines).

FIG. 9. Dendrogram of the 3-ketoacyl-CoA synthase gene family based on the amino acid sequences. The alignment contains the protein sequence of the Crambe abyssinica FAE (CrFAE), compared with those of Brassica juncea FAE1 (BjFAE), Brassica oleracea FAE1 (BoFAE), Brassica napus FAE1 (BnFAE), Arabidopsis thaliana FAE1 (AtFAE) and Tropaeolum majus FAE (TmFAE).

FIG. 10. Hydropathy analysis of Crambe abyssinica FAE. (A) Hydropathy plot of FAE indicating the presence of several hydrophobic regions. (B) Schematic representation of the putative transmembrane domains of C. abyssinica FAE amino-acid sequence as predicted by TMAP analysis [Persson, Argos 1994]. Numbers shown in the boxes correspond to the residues of each membrane domain in FAE.

FIG. 11. Gas chromatogram of fatty acid methyl esters (FAMEs) extracted from yeast cells transformed with A: Crambe abyssinica FAE homolog in pYES2.1/V5-His-TOPO plasmid and B: empty pYES2.1/V5-His-TOPO (control).

FIG. 12. The accumulation of erucic acid (22:1) in T₁ mature seeds of non-transformed Brassica carinata wild-type control (ntB, Black bar) and Brassica carinata transformed with the Arabidopsis FAE1 gene (Lines 2B through 37B; Gray bars).

FIG. 13. The accumulation of 22:1 and VLCFAs in T₂ mature seed of B. napus cv. Hero non-transformed wild-type controls (H-WT) and eight. Hero/FAE1 transgenic lines (H-10-1 to H-20-6). Fatty acid proportions are shown as the % (w/w) of total fatty acids. Each bar represents the mean±S.D. of ten single seed analyses.

FIG. 14. Proportions of erucic acid, total very long chain fatty acids (VLCFA) and oil content in the best seven DH B. napus c.v. Hero/FAE1 transgenic lines and c.v. Hero and elite c.v. Millennium wild-type control plants from field trials. The results represent average±SD of twelve seed samples from ten plants for each transgenic DH line and wild-type controls (WT).

### DETAILED DESCRIPTION OF THE INVENTION

### Example 1

Plant Materials

All experimental lines propagated in the greenhouse were grown at the Kristjanson Biotechnology Complex greenhouses, Saskatoon, under natural light conditions supplemented with high-pressure sodium lamps with a 16 h photoperiod (16 h of light and 8 h of darkness) at 22° C. and a relative humidity of 25 to 30%. Tropaeolum majus plants (cultivar Dwarf Cherry Rose) were grown in the greenhouse and flowers were hand-pollinated. Seeds at various stages of development were harvested, their seed-coats were removed and embryos were frozen in liquid nitrogen and stored at −80° C. Tobacco plants were grown under sterile conditions on MS medium (Murashige and Skoog, 1962) as well as under normal greenhouse conditions. Arabidopsis plants were grown in a growth chamber at 22° C. with photoperiod of 16 h light (120 μE·m⁻²·s⁻¹) and 8 h dark.

Nasturtium Embryo Protein Preparations and Elongase Assays

Embryos (2-3 grams) were ground in a mortar under liquid nitrogen and then 10 ml of IB buffer (80 mM HEPES pH 7.2, containing 2 mM DTT, 320 mM sucrose and 5% PVPP) per g fresh weight was added. The homogenate was filtered through Miracloth and spun for 5 min at 5, 000×g in a Sorvall refrigerated centrifuge at 5° C., the supernatant retained and re-centrifuged at 15,000×g for 25 min. The resulting pellet was resuspended in 80 mM HEPES containing 20% glycerol and 2 mM DTT. The concentration of protein was determined by the BioRad micro-Bradford method. This subcellular fraction was either used directly to determine enzymatic activities or stored at −80° C. until used.

The 15,000×g particulate preparation was used to perform elongation assays as described by Taylor et al., (1992a & b) with the following modifications: The assay mixture consisted of 80 mM HEPES-NaOH, pH 7.2 containing 0.75 mM ATP, 10 μM CoA-SH, 0.5 mM NADH, 0.5 mM NADPH, 2 mM MgCl₂, 200 μM malonyl-CoA, 18 μM [1-¹⁴C] acyl-CoA (0.37 GBq·mol⁻¹) and nasturtium protein in a final volume of 500 μL. The reaction was started by the addition of 200 μg of protein and incubated in a shaking water-bath at 30° C., 100 rpm for 0.5 h. [1-¹⁴C]—Radiolabeled acyl-CoAs were synthesized from the corresponding free fatty acids as described previously by Taylor et al., (1990). Elongase reaction assays were stopped with 3 mL of 100 gL⁻¹ KOH in methanol. Fatty acid methyl esters (FAMEs) were prepared and quantified by radio-HPLC as described previously (Taylor et al., 1992b).

Lipid Analyses

The total fatty acid content and acyl composition of tobacco plant lipids and Arabidopsis seed oils was determined by GC of the FAMEs with 17:0 FAME as an internal standard as described previously (Zou et al., 1997; Katavic et al., 2001; Taylor et al., 2001)

Isolation of Nasturtium FAE cDNA by a Degenerate Primers Approach

Degenerate primers were designed for amino acid sequences conserved among Arabidopsis thaliana KCS1 (AF053345), Brassica napus FAE1 (AF009563), Limnanthes douglasii FAE (AF247134) and Simmondsia chinensis FAE (U37088). Single-stranded cDNA template for reverse transcriptase-PCR was synthesized at 42° C. from embryo poly (A) RNA with PowerScript™ (Clontech). A 50 μL PCR reaction contained single-stranded cDNA derived from 40 ng of poly (A) RNA, 20 pM of each primer: F1-forward TCT(A/T)GG(A/T)GG(C/A)ATGGGTTG (SEQ ID NO:1) [LGGMGC] (SEQ ID NO:2), R1-reverse T(G/A)TA(T/C)GC(C/T)A(A/G)CTC(A/G)TACC (SEQ ID NO:3) [WYELAY] (SEQ ID NO:4) and 2.5 U of Taq DNA Polymerase (Amersham) under standard conditions. An internal part of the elongase sequence was amplified in a thermocycler during 30 cycles of the following program: 94° C. for 30 sec, 48° C. for 30 sec and 72° C. for 1 min. The sequence of a 650-bp PCR product was used to design a primer to amplify the 5′ and 3′ ends of the cDNA using the SMART™ RACE cDNA Amplification Kit (CLONTECH). After assembly to determine the full length sequence of the cDNA, the open reading frame (ORF) was amplified using the primers P-forward ACCATGTCAGGAACAAAAGC (SEQ ID NO:5) and PR-reverse TTAATITAATGGAACCTCAACCG (SEQ ID NO:6), and subsequently cloned into the pYES2 expression vector (Invitrogen).

cDNA Library Construction

To construct the nasturtium developing cDNA library, immature seeds were collected 17 days after pollination. Total RNA was extracted from embryos according to Lindstrom and Vodkin (1991), then poly (A) RNA was isolated using Dynabeads Oligo (dT)₂₅ (DYNAL). Copy DNA synthesis was performed on 1 μg of poly (A) RNA using SMART PCR cDNA Synthesis Kit (Clontech) according to manufacturer's protocol. The cDNA population was then subtracted with 12S and 2S seed storage protein cDNA clones using PCR-Select cDNA Subtraction Kit (Clontech). The subtracted embryo cDNA population was cloned and then sequenced as described by Jako et al. (2002).

Sequence Handling

Sequence analyses were performed using Lasergene software (DNAStar). Sequence similarity searches and other analyses were performed using BLASTN, BLASTX and PSORT programs.

Site Directed Mutagenesis of FAE

A site-directed mutagenesis experiment was performed essentially as described previously (Katavic et al., 2002). The desired mutation (tyrosine at position 429 is replaced with histidine) was introduced into the FAE coding region by polymerase chain reaction using primers F2-forward TCGAGGATGTCGCTTCACCGATTTGGAAACAC (SEQ ID NO:7) and R2-reverse GTTTCCAAATCGGTGAAGCGACATCCTCGATGG (SEQ ID NO:8). Primers were complementary to the opposite strands of pYES2.1/V5His-TOPO containing the nasturtium FAE gene.

Northern Analysis

Total RNA from nasturtium plant material was isolated according to Lindstrom and Vodkin (1991). 20 microgram of RNA was fractionated on a 1.4% formaldehyde-agarose gel and the gels were then stained with ethidium bromide to ensure that all lanes had been loaded equally. The RNA was subsequently transferred to Hybond N⁺ membrane and hybridized with the ³²P labeled FAE DNA probe, prepared using the Random Primers DNA labeling kit (Gibco-BRL, Cleveland). Membranes were hybridized at 60° C. overnight.

Plant Transformation Vectors

The coding regions of the nasturtium FAE (natural and mutated versions named SF and SMF, respectively) were amplified by polymerase chain reaction with primers F3-forward: taggatccATGTCAGGAACAAAAGC (lower case indicates the restriction site for BamHI) (SEQ ID NO:9); and R3-reverse tagagctcTTAATTTAATGGAACCTCAACC (lower case indicates the restriction site for SacI enzyme) (SEQ ID NO:10) and subsequently cloned as a BamHI and SacI fragment behind the constitutive 35S promoter in binary vector pBI121 (CLONTECH). The coding region of the nasturtium FAE was cloned behind the seed-specific napin promoter as follows: A BamHI site was introduced in front of the start codon and behind the stop codon of FAE by PCR with primers F3 (as above) and R4-reverse: taggatccTTAATTTAATGGAACCTCAACC (lower case indicates the restriction site for BamHI) (SEQ ID NO:11). The B. napus napin promoter was cloned in HindIII/XbaI sites of the pUC19 (Fermentas) and the nos terminator was introduced as an EcoRI/BamHI fragment. The resulting vector was named pDH1. The napin promoter/nos terminator cassette was excised by HindIII/EcoRI digestion and subsequently cloned into the respective sites of pRD400 (Clontech) resulting in pVK1. The coding region of FAE was then cloned into the BamHI site of pVK1 behind the napin promoter and the resulting vector was named NF. Sense orientation of the FAE coding region with respect to the promoter was confirmed by restriction analyses with XbaI.

The final binary vectors (SF: 35S-FAE, SMF: 35S-Mutated FAE, and NF: napin: FAE) were electroporated into Agrobacterium tumefaciens cells strain GV3101 containing helper plasmid pMP90. Plasmid integrity was verified by DNA sequencing following its re-isolation from A. tumefaciens and transformation into E. coli.

Plant Transformation and Genetic Analysis

Tobacco (Nicotiana tabacum cv. Xanthi) was transformed using a leaf disc transformation procedure (Horsch et al., 1985). Shoots that rooted in the presence of 50 μg/mL kanamycin were considered to be transgenic. Transgenic plants were transferred to soil and grown in the greenhouse.

Arabidopis (A. thaliana ecotype Wassilewskija) were transformed by vacuum infiltration according to the method of Clough and Bent (1998). Transgenic plants were selected and analyzed essentially as described by Jako et al., (2001).

Molecular Analysis of Transgenic Plants

DNA was isolated from 2-3 g of tobacco or 150 mg of Arabidopsis leaf material using a urea-phenol extraction method (Chen et al., 1992) with the following minor modification: Material was frozen in liquid nitrogen and kept at −80° C. until used. Extraction was performed for 15 min at room temperature and 400 mM ammonium acetate, pH 5.2 was used for the first two precipitation steps. Stable integration of the napin:FAE:nos cassette into the genome of transgenic plants was checked by PCR amplification on genomic DNA with NN3 and NN4 primers as described by Katavic et al., (2001).

Southern analyses were performed to further confirm and select those transformants containing single or multiple copies of the inserted fragments. 15 microgram of tobacco or 1 microgram of Arabidopsis genomic DNA was digested with the restriction enzyme SacI, and the resulting fragments were separated on a 0.9% (w/v) agarose gel, transferred to Hybond N⁺ nylon membrane (Amersham) via an alkali blotting protocol. A 1.5 Kbp probe containing the coding sequence of FAE was generated by polymerase chain reaction (PCR) using primers: F4-forward ATGTCAGGAACAAAAGC (SEQ ID NO:12) and R5-reverse TAATTTAATGGAACCTCAACCG (SEQ ID NO:13) and subsequently radioactively labeled with ³²P as described above. Hybridization was performed at 65° C. The filters were washed once in 1×SSPE, 0.1% SDS for 15 min and in 0.1×SSPE, 0.1% SDS for 5-10 min at the temperature of hybridization. The blots were developed by exposure to X-OMAT-AR film (Kodak, Rochester, N.Y.).

To estimate the number of FAE isoforms in the T. majus genome, 15 microgram of genomic DNA was digested with restriction enzymes: EcoRI, AccI, NcoI and HindIII. Blotting and hybridization conditions were essentially as above except that filters were washed at low stringency with 1×SSPE, 0.1% SDS for 15 min, autoradiographed and then washed subsequently with 0.1×SSPE, 0.1% SDS, and re-exposed.

### Example 2

Acyl Composition of T. majus cv Dwarf Cherry Rose

The acyl composition of the TAG fraction of this cultivar was typical in that it had highly enriched proportions of very long chain monounsaturated fatty acids (VLCMFAs), particularly 22:1 (77.5%) and 20:1 (16.0%) with a trace of 24:1 (1.5%), and a low proportion of total C₁₈ fatty acids (2.5%), primarily 18:1 (2.4%). The predominant TAG species were trierucin followed by 22:1/20:1/22:1 (Taylor et al., 1992a).

### Example 3

Substrate Specificity of Nasturtium Embryo Elongases In Vitro

Although there has been considerable debate regarding the acyl substrate for elongase activity in developing oilseeds, recent studies of developing seeds of B. napus have revealed the presence of two types of elongation activity in vitro: an acyl-CoA-dependent activity, and an ATP-dependent activity which apparently utilizes an endogenous acyl primer. A 15,000×g particulate fraction was isolated from nasturtium embryos collected at mid-development (at 14-17 days after pollination), the stage which exhibited the highest enrichment in acyl-CoA-dependent elongase activity.

It has been shown that while ATP is necessary for acyl-CoA-dependent elongation in vitro, too high a concentration of ATP strongly inhibited elongase activity. In addition, elongase enzyme activity has been reported to be stimulated by the presence of 10 μM CoASH. In order to optimize reaction conditions, we assessed the roles of these two co-factors. Elongase activity was measured in vitro in the 15,000×g particulate fraction from nasturtium embryos under different ATP concentrations (0-5 mM) in the presence of 10 μM CoASH with 18 μM 1-[¹⁴C]-18:1-CoA and 200 μM malonyl-CoA. The highest activity was found at a concentration of 0.75 mM of ATP. Then, elongase activity was examined with range of [1-¹⁴C]-acyl-CoAs substrates at an ATP concentration of 0.75 mM in the presence of 10 μM CoASH.

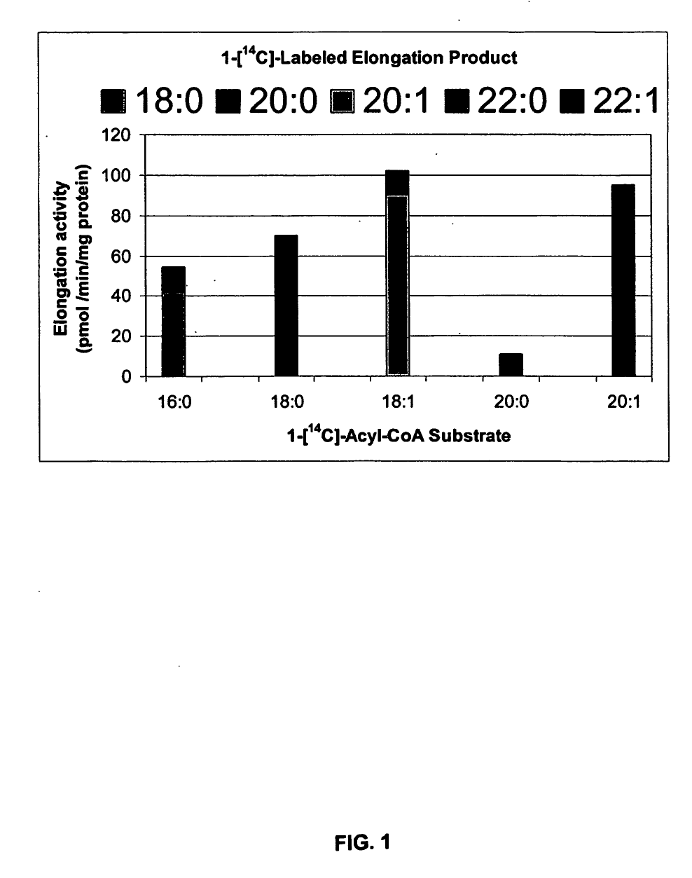

Our results indicate that in a developing nasturtium embryo particulate fraction, acyl-CoA-dependent elongases have the capacity to elongate a wide range of saturated (C₁₆-C₂₀) and monounsaturated (C₁₈ and C₂₀) fatty acyl moieties (FIG. 1). Of the [1-¹⁴C]-labeled acyl-CoA series (16:0-CoA, 18:0-CoA, 18:1-CoA, 20:0-CoA, 20:1-CoA, 22:1-CoA), tested in vitro, elongase(s) from mid-developing nasturtium embryos exhibited the highest activity with 18:1-CoA and 20:1-CoA (102 and 95 pmol/min/mg protein, respectively). These elongase activity rates are of the same order of magnitude as that reported for acyl-CoA elongase(s) in a similar particulate fraction from developing rapeseed embryos. The particulate fraction was also able to elongate, in order of specificity, the saturated substrates 18:0-CoA, 16:0-CoA, and to a much lesser extent, 20:0-CoA. In general, regardless of the 1-[¹⁴C]acyl-CoA substrate supplied in vitro, the major labeled fatty acyl product was the C₂ extension of its respective precursor (about 80-90%), with the next respective C₄ extension product being present in proportions of about 10-20% (FIG. 1). The one critical exception to this trend was the production solely of radiolabeled erucic acid from its respective 1-[¹⁴C] eicosenoyl-CoA precursor. There was no detectable elongation of 1-[¹⁴C]-labeled 22:1-CoA to 24:1, even though the latter is found in trace amounts in nasturtium seed oil.

### Example 4

Isolation of T. majus FAE Homolog

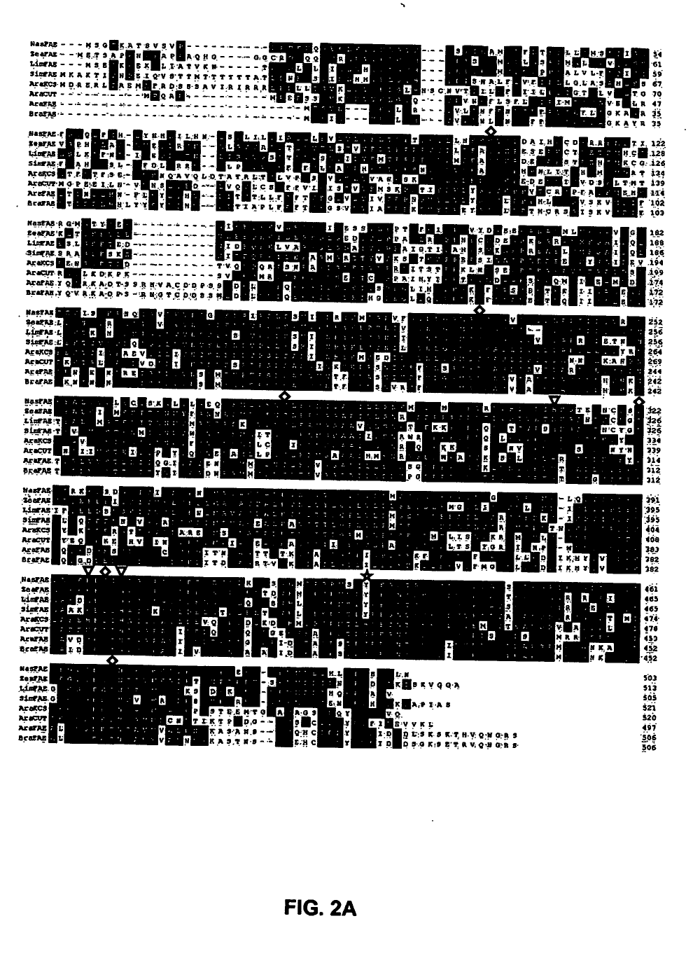

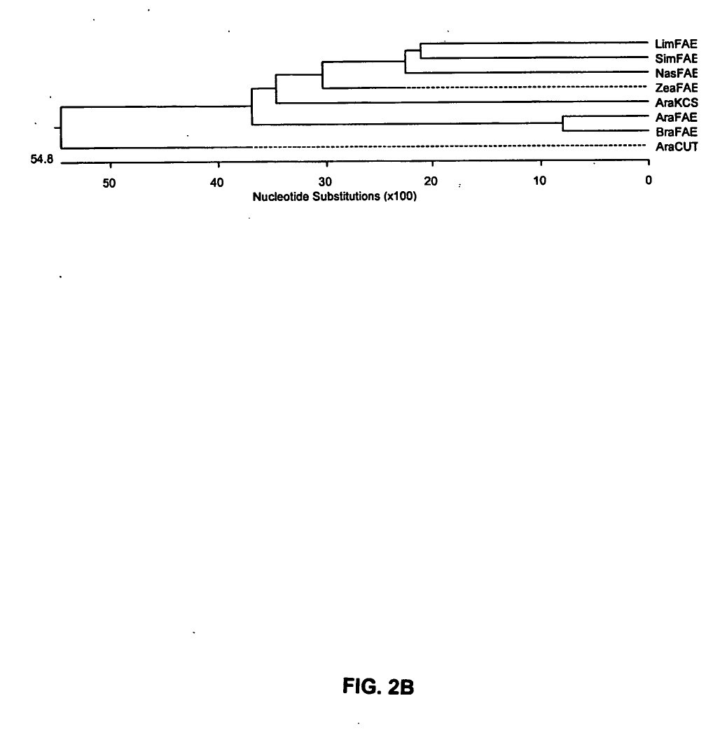

Based on sequence homology among plant fatty acid elongase genes, a full-length clone was amplified by PCR using a degenerate primers approach and the sequence submitted to the GenBank (accession number AY082610; FIG. 2 (A)). The nucleotide sequence had an open reading frame of 1512 bp. Subsequently, 3 partial clones of about 0.6 kb, representative of the AY083610 FAE clone, were found among 2,800 ESTs surveyed (about 0.1% representation) from a nasturtium embryo subtracted cDNA library.

Alignment of the amino acid sequence of the nasturtium FAE with other plant condensing enzymes revealed the presence of six conserved cysteine residues (FIG. 2A.). Further sequence analysis showed that one out of the four conserved histidine residues suggested to be important for Arabidopsis FAE1 activity, was substituted with tyrosine in the T. majus FAE polypeptide.

An analysis of the nucleotide sequence of the corresponding nasturtium FAE genomic clone revealed the absence of intron sequences. A similar absence of introns was observed in homologs from

A. thaliana FAE1, rapeseed CE7 and CE8 and high and low erucic lines of B. oleracea, B. rapa, canola B. napus cv Westar and HEAR B. napus cv Hero.

The T. majus FAE cDNA encodes a polypeptide of 504 amino acids that is most closely related to an FAE2 from roots of Zea mays (69% amino acid identity) (FIG. 2 (B)). The T. majus FAE polypeptide also shared strong identity with FAEs from Limnanthes douglasii (67%) and from seeds of jojoba (Simondsia chinensis) (63%). Homology of the nasturtium FAE to two Arabidopsis 3-ketoacyl-CoA synthases AraKCS and AraCUT1) involved in cuticular wax synthesis was on the level of 57% and 53%, respectively. These homologs all exhibit the capability to elongate saturated fatty acids to produce saturated VLCFAs. The FAE1 polypeptides involved in the synthesis of VLCFAs in Arabidopsis and Brassica seeds showed approximately 52-54% identity with the T. majus FAE. The nasturtium FAE protein was predicted to have a theoretical pI value of 9.3 using the algorithm of Bjellqvist et al., (1993 and 1994) and a molecular mass of 56.8 kDA, which are similar to the respective values reported for the B. napus CE7 and CE8 FAE homologs as well as those from B. rapa (campestris) and B. oleracea.

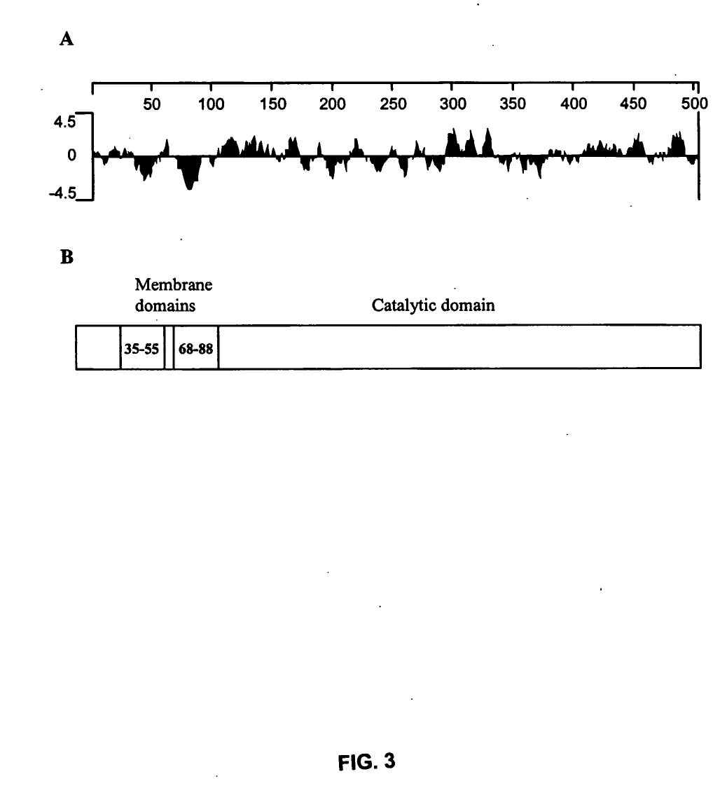

A hydropathy analysis (Kyte-Doolittle) of the amino acid sequence of the T. majus FAE revealed several hydrophobic domains (FIG. 3A). Protein analyses with the TMAP algorithm (Person and Argos, 1994) predicted two N-terminal transmembrane domains, the first corresponding to amino-acid residues 35-55, and the second spanning residues 68-88 (FIG. 3B). Like other elongase condensing enzymes, the T. majus FAE lacks N-terminal signal sequences typically found for plastidial or mitochondrial-targeted plant enzymes. It also lacks a KXKXX or KKXX motif (X=any amino acid) often found at the C-terminus of proteins retained within ER membranes. Rather, it is a type IIIa protein, typically present on endoplasmic reticular membranes.

### Example 5

Tissue Specific Expression and Copy Number Estimate of T. majus FAE

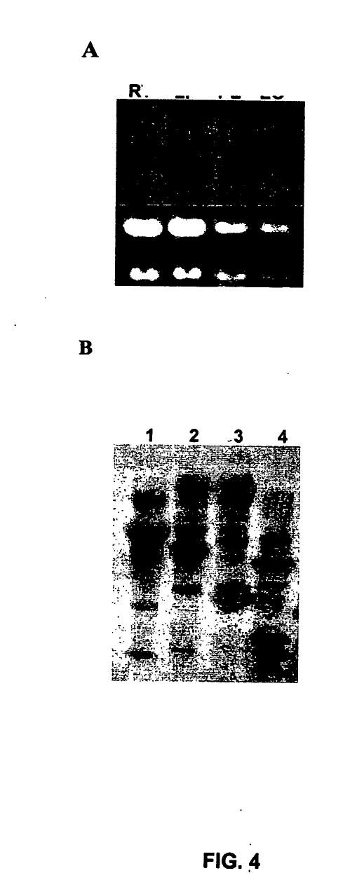

Northern blot analyses were performed to investigate the expression profile of the FAE gene. Total RNA was isolated from different nasturtium tissues including roots, leaves, floral petals and mid-developing embryos. A strong hybridization signal with FAE-specific probe was observed only with RNA isolated from developing embryos (FIG. 4. A).

A Southern blot hybridization was performed with nasturtium genomic DNA digested with several restriction enzymes including EcoRI, AccI, NcoI and HindIII. The FAE gene has no internal EcoRI, AccI or NcoI sites, while one internal HindIII site exists. Autoradiography revealed the presence of one strongly-hybridizing fragment in all cases except with HindIII for which two strongly hybridizing fragments were evident (FIG. 4.B). In addition a minimum of 4 weakly hybridizing fragments were detected. After washing under high stringency conditions, the number of hybridizing fragments was unchanged. Thus, we have concluded that T. majus FAE belongs to a multigenic family consisting of 4 to 6 members. A similar multigenic family has been found for a rapeseed FAE1 gene member.

### Example 6

Heterologous Expression of the T majus FAE in Yeast

To study the function of the protein encoded by the T. majus FAE, the coding region was linked to the galactose-inducible GAL1 promoter in the expression vector pYES2 and transformed into yeast. Transgenic yeast cells harbouring the T. majus FAE did not show any difference in fatty acid composition in comparison to yeast cells transformed with empty vector. A similar difficulty with expression of Limnanthes FAE and corn FAE in yeast cells has been reported.

As indicated earlier, a comparison of the predicted amino acid sequence of the nasturtium FAE with other plant condensing enzymes (FIG. 2A) showed that one of the four conserved histidine residues, known suggested to be important for Arabidopsis FAE1 activity, was substituted with tyrosine in the T. majus FAE polypeptide. To study the importance of this histidine residue for enzyme activity, we used a site directed mutagenesis approach to replace the tyrosine 429 residue with histidine. This mutated version of nasturtium FAE was expressed in yeast cells. Analyses of fatty acid composition of transformed yeast cells showed that histidine at position 429 did not restore enzyme activity. Therefore we decided to study the function of T. majus FAE in plant heterologous chromosomal backgrounds.

### Example 7

Expression of T. majus FAE in Tobacco Plants

To establish functional identity, the cDNA for the FAE-related polypeptides was constitutively expressed in tobacco plants under the control of the tandem 35S constitutive promoter. In addition, to assess the importance of histidine residues for enzyme activity, the tyrosine at position 429 in the nasturtium FAE was replaced with histidine and subsequently used to prepare a plant transformation vector under the control of the tandem 35S promoter. Integration of the 35S/FAE/Nos expression cassette into tobacco plants was confirmed by PCR amplification on genomic DNA. Fatty acid composition was determined in callus, leaves and seeds of transgenic tobacco plants.

Constitutive expression of the nasturtium FAE homologue in tobacco callus resulted in an increase in proportions of VLCFAs from 3.7% in the wild type background to as high as 8.6% (a 132% increase) in transgenic lines (Table I). In particular, the increase in proportions of saturated VLCFAs (22:0, 24:0, and 26:0) was most pronounced. The fact that the synthesis of the saturated VLCFAs occurs at the expense of 16:0 and 18:0 suggests that the nasturtium FAE is able to elongate C₁₆ and C₁₈ fatty acids. Expression of the mutated version of the nasturtium FAE (SMF) resulted in a slight increase in the VLCFA content in tobacco callus, on average 18.5% in comparison to the wild type background. Increased proportions of VLCFAs at the expense of LCFAs were observed in leaves of transgenic tobacco plants carrying either the nasturtium FAE or its mutated form (Table II). Comparison of fatty acid composition in tobacco tissues upon expression of the nasturtium FAE and its mutated version, revealed that tyrosine at position 429 is likely important to achieve full activity of the enzyme. A decreased proportion of 18:3 in leaves of transgenic tobacco lines in comparison to the wild type (empty vector) background suggests that the “metabolic pull” of the elongation pathway may be somewhat stronger than that of the competing desaturation pathway.

Expression of nasturtium FAE in tobacco seeds resulted in a 50% increase in proportions of VLCFAs from 0.6% in the wild type background to 0.9% in transgenic plants (data not shown). The relatively low proportions of VLCFAs in tobacco leaves and callus (see Tables I and II) may be an indication that (i) in vivo, saturated fatty acids are not present at high concentrations; therefore even a 50% increase in relative proportions does not result in high levels of VLC saturated fatty acids accumulating in glycerolipids; (ii) expression of the nasturtium FAE when under the control of the 35S promoter is relatively weak.

### Example 8

Expression of T. majus FAE in Arabidopsis Seeds

Since expression of nasturtium FAE under the control of the 35S promoter did not result in a high accumulation of VLCFA in tobacco seeds we decided to study the effect of expressing it in Arabidopsis. Using a vacuum-infiltration method, 18 kanamycin resistant Arabidopsis plants were obtained. The fatty acid composition of T₂ seeds was determined. A significant increase was observed only in the content of erucic acid (22:1 c13). On average, the level of erucic acid increased up to 3.2% (a 50% increase) in transgenic seeds comparing to 2.1% in wild type background (data not shown). In the best transgenic lines, the content of erucic acid increased up to 4.0% (a 90% increase).

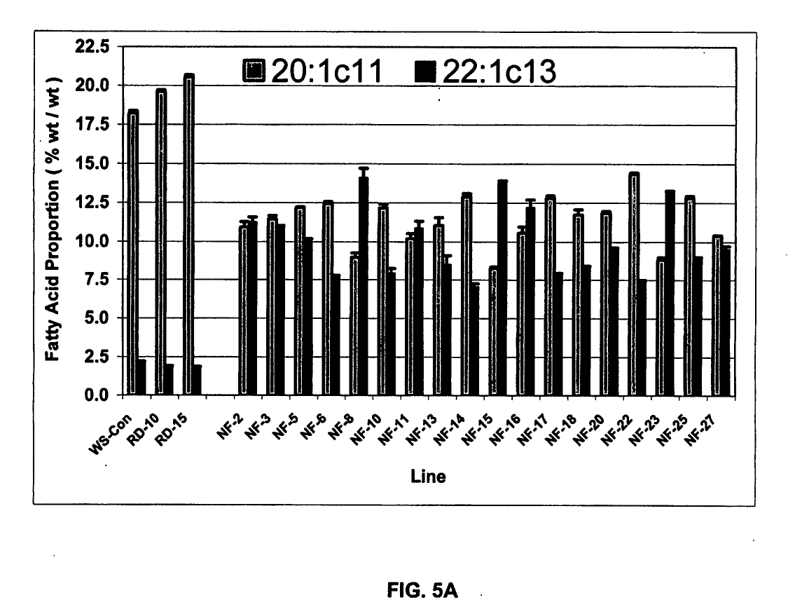

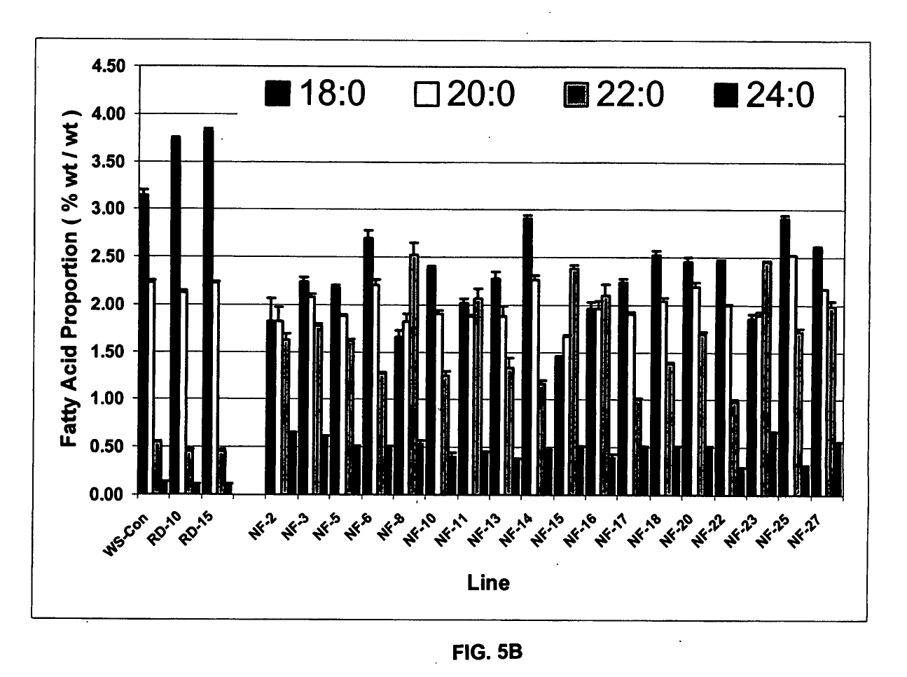

Since tandem 35S-driven constitutive FAE expression did not result in a strong increase in VLCFA proportions in tobacco and Arabidopsis seeds, we decided to use the seed-specific promoter napin to study FAE expression in an Arabidopsis seed background. From vacuum-infiltration experiments, 25 kanamycin-resistant T₁ plants were selected. The T₂ progeny were collected individually from each plant and the fatty acid composition determined. Significant changes in fatty acid composition in comparison to the wild type (empty vector) were found. On average, the proportion of erucic acid (22:1 Δ13) increased from 2.1% in wild type to 9.6% in T₂ transgenic seeds at the expense of 20:1 Δ11 (Table III). Eighteen of the best transgenic lines were selected to examine the range of VLCFA proportional re-distribution induced by expression of the nasturtium FAE gene (FIGS. 5A and B). The erucic acid content was increased by up to 6.5-fold in line NF-8. Small increases in the proportions of 24:1 Δc 15 were also observed (Table III). There was also a relatively significant increase in the proportions of the saturated VLCFAs, 22:0 and 24:0, at the expense of 18:0 and 20:0. In both the case of the VLC mono-unsaturated fatty acids (FIG. 5A) and the VLC saturated fatty acids (FIG. 5B), the highest proportional increases in erucic and in [behenic+lignoceric] acids were generally correlated with the concomitant reduction in the proportion of their corresponding elongase primers, eicosenoic and [stearate+arachidic] acids, respectively.

Therefore, we conclude that the nasturtium FAE is able to preferentially elongate 20:1 and [18:0+20:0]. As would be expected, there was significant variation in the proportions of 22:1 which accumulated (FIG. 5A) possibly due to positional effects from nasturtium FAE transgene insertion at different sites in the Arabidopsis genome. Similar variations were observed in the expression of a castor fatty acid hydroxylase gene (CFAH12) in the Arabidopsis fad2/fae1 mutant.

In summary, we have isolated a cDNA clone from nasturtium which exhibits high similarity to the sequences of 3-ketoacyl-CoA synthases from various plant species but has the unprecedented capability to increase the erucic acid content by 8-fold in Arabidopsis thaliana.

Our in vitro findings suggest that the FAE proteins in a 15,000×g nasturtium particulate fraction have a broad acyl-CoA preference, with the ability to elongate both monounsaturated and saturated C₁₈-CoA and C₂₀-CoA substrates. In like manner, a partially purified jojoba FAE1 showed maximal activity with monounsaturated and saturated C₁₈ and C₂₀-CoAs in vitro. However, it is important to note that the particulate elongation activity reported in the current study most likely represents the cumulative effect of expression of more than one member of this small gene family. Thus, from this experiment one can only conclude that the capacity to elongate both monounsaturated and saturated acyl moieties is represented in this nasturtium particulate fraction.

While genetic analyses and homology assessments might predict that the isolated nasturtium FAE gene might encode an enzyme which prefers to elongate saturated acyl-CoAs, the transgenic experiments in tobacco callus, tobacco leaves and in Arabidopsis seed, collectively confirmed that the heterologously-expressed T. majus FAE gene product can elongate both monounsaturated and saturated acyl moieties. In fact, in a transgenic Arabidopsis background, the nasturtium FAE was much stronger than the jojoba 3-KCS in its ability to increase the level of 22:1: Introducing the jojoba cDNA into Arabidopsis resulted in an increase in 22:1 proportions from about 2% in the control to 4% in the transgenics. In comparison, when we introduced the T. majus FAE into Arabidopsis, the erucic acid content increased by almost an order of magnitude (8-fold) at the concomitant expense of 20:1 Δ11. The acyl composition of the transgenic Arabidopsis seed oil was reproportioned such that erucic and eicosenoic became about equal as the two predominant VLCFAs.

The ability of the nasturtium FAE protein to preferentially elongate 18:1-CoA and especially 20:1-CoA, is consistent with the observed acyl composition of nasturtium seed oil which consists primarily of very long chain- and specifically erucoyl moieties. We postulate therefore, that whether the nasturtium FAE transgene results in predominantly mono-unsaturated (20:1 Δ11, 22:1 Δ13) or saturated (e.g. 20:0, 22:0) VLCFAs is more a function of the composition of the acyl-CoA pool (18:1 Δ9 and 20:1 Δ11 or 18:0 and 20:0 or, respectively) available to the condensing enzyme in the host species/target organ.

Thus, the nasturtium FAE homolog described herein, will have a larger engineering impact when strongly expressed in a seed-specific manner in H.E.A. Brassicaceae (e.g. B. napus; B. carinata) wherein 18:1 Δ9 [and 18:2/18:3] and 20:1 Δ11 represent a potential acyl-CoA elongation substrate pool of almost 40% to enrich the already-existing 22:1 Δ13 content. Clearly, the current studies indicate that the nasturtium FAE expression should be combined with other genetic modifications we have made to enhance the VLCFA content of HEAR Brassicaceae and the proportions of erucic acid in particular, to provide an industrial feedstock oil of high value and broad applicability.

A major goal of our research is to obtain, by genetic manipulation, Brassica napus L. cultivars or B. carinata breeding lines with higher levels of erucic acid (22:1) in their seed oil than in present Canadian HEA cultivars. For example, we would like to develop a B. napus cultivar containing erucic acid levels above 66 mol %, ideally with more than 80% erucic acid in the seed oil. To achieve our goals we are isolating new, more efficient strategic genes for high erucic acid and preferably, trierucin, production. We selected Tropaeolum majus, garden nasturtium, as a source of those genes based on the fact that this plant is capable of producing significant amounts of erucic acid (70-75% of total fatty acid) and accumulates trierucin as the predominant TAG in its seed oil. The fatty acid elongase (FAE), 3-ketoacyl-CoA synthase (KCS) is the first component of the elongation complex involved in synthesis of erucic acid (22:1) in seeds of Tropaeolum majus (garden nasturtium). Using a degenerate primers approach, a cDNA of an embryo FAE was obtained and heterologously expressed in two different plant backgrounds (A. thaliana and N. tabacum) under the control of a seed-specific (napin) promoter and the constitutive (tandem 35S) promoter, respectively. Seed-specific expression resulted in up to an 8-fold increase in erucic acid proportions in Arabidopsis seed oil. Constitutive expression in transgenic tobacco tissue resulted in increased proportions of very long chain saturated fatty acids. These results indicate that the nasturtium FAE gene encodes a condensing enzyme involved in the biosynthesis of very-long chain fatty acids, utilizing monounsaturated and saturated acyl substrates. Thus, the nasturtium FAE homolog will have a larger engineering impact when strongly expressed in a seed-specific manner in H.E.A. Brassicaceae (e.g. B. napus; B. carinata) wherein 18:1 Δ9 [and 18:2/18:3] and 20:1 Δ11 represent a potential acyl-CoA elongation substrate pool of almost 40% over and above the existing high 22:1 Δ13 content.

In addition, heterologous expression of the nasturtium FAE gene in HEAR Brassicaceae can be combined with other genetic modifications we have made to enhance the VLCFA content of HEAR germplasm (Katavic et al., 2001; Taylor et al., 2001) and the proportions of erucic acid in particular, to provide an industrial feedstock oil of high value and broad applicability.

Expression of nasturtium FAE in Arabidopsis seeds resulted in an 8-fold increase in erucic acid content. Therefore, it is anticipated that the introduction of this gene alone, or in combination with other altered gene expression phenotypes (e.g. FAE1 and/or FAD2 and/or FAD3) into HEAR Brassicaceae will result in transgenic lines with improved proportions of erucic acid in the seed oil.

### Example 9

Heterologous Expression of the Nasturtium FAE in HEAR Brassicaceae—e.g. B. carinata

The nasturtium FAE gene under the control of the strong seed-specific promoter napin, was introduced into HEAR Brassicaceae (e.g. B. carinata). Considering the results obtained in Arabidopsis seeds, we anticipated that there would be a strong increase in the proportion of 22:1 and saturated VLCFAs as well (by up to 8-10%).

The coding region of nasturtium FAE was cloned behind a napin promoter as described in Example 1. The resulting plasmid named NF was electroporated into Agrobacterium tumefaciens and subsequently used to transformed Brassica carinata plants using the protocol described by Babic et. al., (1998). Shoots that rooted in the presence of 25 mg/L kanamycin were considered to be transgenic. Transgenic plants were transferred to soil and grown in the growth chamber. T₁ seed from self pollinated plants were harvested and subjected to biochemical analysis performed as described by Mietkiewska et al (2004). The proportion of erucic acid increased from 30% in wild type controls to as high as 39% in T₁ segregating seeds of the best transgenic line (FIG. 6).

### Example 10

Heterologous Co-Expression of Two FAE Genes in HEAR Brassicaceae (e.g. B. carinata) Co-Transformed with the Napin:AthalFAE1+Napin:NastFAE

Expression of nasturtium FAE in HEAR Brassicaceae (e.g. B. carinata) and the resulting proportional increase in erucic acid can be maximized by also addressing the fact that 20:1, the preferred monounsaturated substrate, is present in wild type seeds in relatively low proportions (5.5-6.5%). Therefore, for example, one can introduce the Arabidopsis FAE1 and nasturtium FAE into HEAR Brassicaceae (e.g. B. carinata). The first gene product should enhance conversion of 18:1 to 20:1 (Katavic et al., 2001), while the nasturtium FAE gene product clearly prefers to extend 20:1 to 22:1. In this manner, the maximal proportion of erucic acid is expected. To achieve this goal, one could apply a co-transformation method: The Arabidopsis FAE is cloned in a derivative of vector pRD400 which allows selection on kanamycin, while the nasturtium FAE is cloned in pCAMBIA vector which allows selection on hygromycin.

The coding region of the nasturtium FAE with the nos terminator were excised from the SF plasmid (Mietkiewska et al., 2004) by BamHI/EcoRI digestion. The napin promoter was excised from the NF plasmid (Mietkiewska et al., 2004) by a HindIII/BamHI restriction reaction. Isolated fragments were cloned in HindIII/EcoRI sites of pCAMBIA1390 and the resulting plasmid was named NFPC. The binary vector (AFAE) containing the Arabidopsis FAE1 under the control of the napin promoter was kindly provided by Dr L. Kunst, University of British Columbia, Vancouver, BC Canada and was constructed as described by Katavic et al, 2000 and by Katavic et al., 2001.

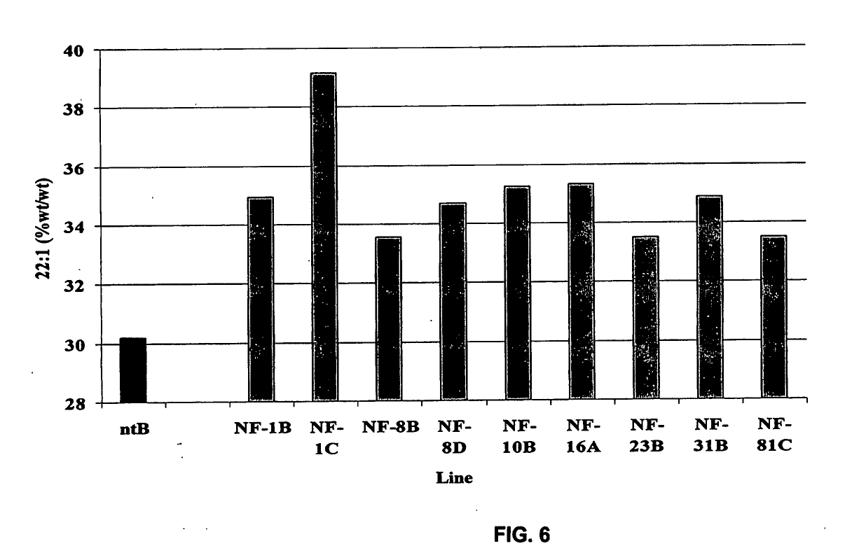

The binary vectors (NFPC, AFAE) were electroporated into Agrobacterium tumefaciens and subsequently introduced into Brassica carinata plants in a co-transformation experiment. Double transformants were selected on media supplemented with both kanamycin (25 mg/L) and hygromycin (10 mg/L). The selected plants were grown in the growth chamber. T₁ seeds were collected and subjected to biochemical analysis. As shown in FIG. 7, significant changes in the content of erucic acid were found in the double transformants compared to the control. The proportion of erucic acid increased from 30% in wild type to as high as 44-46% in T₁ segregating seeds of the best transgenic lines.

### Example 11

Heterologous Co-Expression of the Napin: NastFAE and the Limnanthes Des 5 Desaturase Genes Co-Transformed into Arabidopsis thaliana

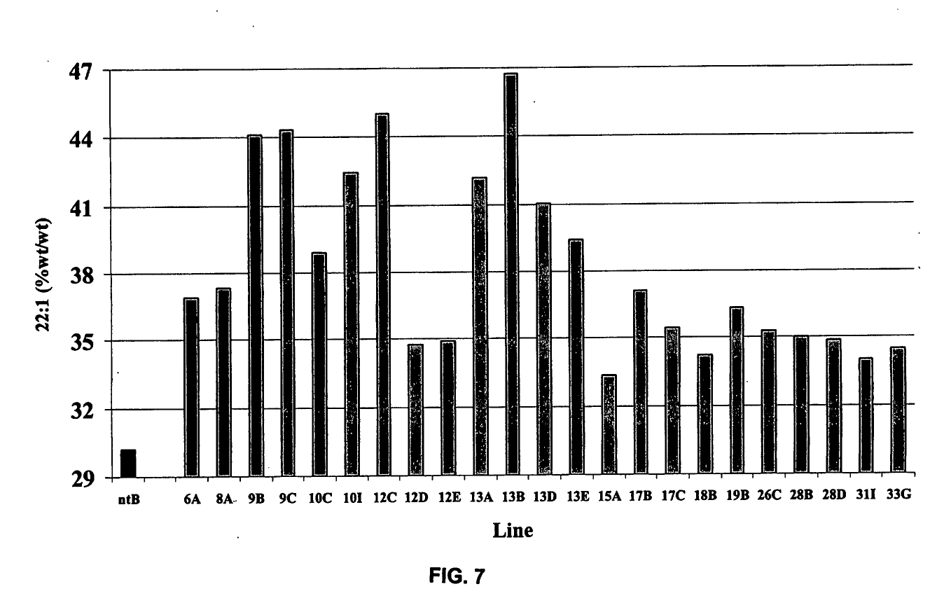

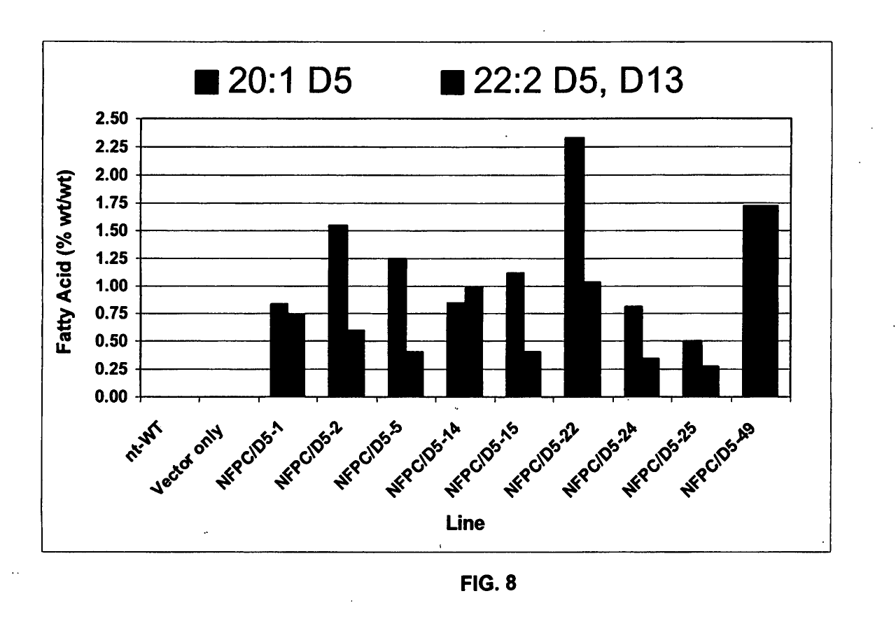

The coding region of nasturtium FAE behind the napin promoter in pCAMBIA1390 vector (NFPC) was cloned as described in Example no. 10. The coding region of Limnanthes Δ5 desaturase was cloned behind the napin promoter as follows: The plant transformation vector pSE129A, already prepared from pRD400 plasmid (Datla et al., 1992), was obtained by introducing a HindIII/XbaI fragment containing the B. napus napin promoter and a KpnI/EcoRI fragment containing the Agrobacterium nos terminator. The 1.0 kb open-reading frame of the Limnanthes Des5 (Limnanthes Acyl-CoA Δ5 desaturase, GenBank Accession no AF247133) was amplified by PCR with primers designed to contain XbaI and KpnI restriction sites and was ligated into XbaI/KpnI-digested pSE129A in the sense orientation to give the binary vector Limdes5/pSE. The binary vectors NFPC and Limdes5/pSE were introduced into Agrobacterium tumefaciens cells strain GV3101 as described in Example no. 10. was then introduced by electroporation into Agrobacterium tumefaciens strain GV3101 bearing the helper plasmid pMP90 for plant transformation. Arabidopsis plants (A. thaliana ecotype Wassilewskija) were co-transformed by the vacuum infiltration method (Clough and Bent, 1998) using equal volumes of the two recombinant A. tumefaciens suspensions. Transgenic plants were selected and analyzed as described by Mietliewska et al., (2004). Seeds (T₁) obtained from co-transformed plants were selected on media supplemented with both kanamycin (50 mg/l) and hygromycin (20 mg/l). The selected plants were grown in the growth chamber. T₂ seeds were collected and subjected to biochemical analysis (See FIG. 8).

### Example 12

Cloning of Crambe abyssinica 3-Ketoacyl-CoA Synthase (FAE) and Heterologous Expression in Yeast.

Based on FAE1 sequences from Arabidopsis thaliana and Brassica napus, the forward primer F5 (5′-GCAATGACGTCCATTAACGTAAAG-3′) (SEQ ID NO:14) and the reverse primer R6 (5′-TTAGGACCGACCGTTTTGGGC-3′) (SEQ ID NO:15) were designed and used to isolate the FAE coding region from Crambe abyssinica. Genomic DNA isolated from leaves according to urea-phenol extraction method (Chen et al., 1985) was used as a template for PCR amplification with Vent DNA polymerase (New England Biolabs) in a thermocycler during 30 cycles of the following program: 94° C. for 30 sec, 56° C. for 30 sec, and 72° C. for 2 min. A 1.5-kB PCR product was cloned into pYES2.1/V5-His-TOPO expression vector and subsequently sequenced. The Crambe FAE in pYES2.1V5-His-TOPO was transformed into Saccharomyces cerevisiae strain Inv Sci (Invitrogen) using the S.c. EasyComp transformation kit (Invitrogen). Yeast cells transformed with pYES2.1/V5-His-TOPO plasmid only were used as a control. The transformants were selected and grown as described previously (Katavic et al., 2002; Mietkiewska et al., (2004). Fatty acid methyl esters (FAMEs) from yeast cultures were extracted and analyzed as described by Katavic et al., (2002).

### Example 13

Crambe FAE Sequence Handling

Sequence analyses were performed using Lasergene software (DNAStar, Madison, Wis., USA). Sequence similarity searches and other analyses were performed using BLASTN, BLASTX and PSORT programs.

### Example 14

Isolation of Crambe abyssinica FAE Homolog

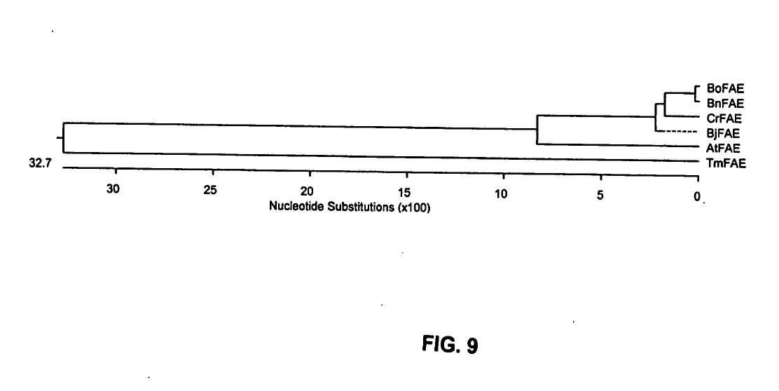

Based on the sequence homology among plant fatty acid elongase genes, a coding region of the FAE gene from Crambe abyssinica was isolated and the sequence submitted to GenBank (Accession no. AY793549). The Crambe FAE open reading frame of 1521-bp encodes a polypeptide of 506 amino acid that is most closely related to an FAE1 from Brassicaceae (FIG. 9): B. juncea (97% identity, GenBank # AJ558198), B. oleracea (96% identity, GenBank # AF490460), B. napus (96% identity, GenBank #AF490459) and B. rapa (96% identity, GenBank #AF49041). The Arabidopsis FAE1 (GenBank # U29142) polypeptide showed 84% identity with the Crambe FAE. Previously isolated Tropaeolum majus FAE (GenBank #AY082610) showed 54% identity with the Crambe FAE. The Crambe FAE protein was predicted to have a molecular mass of 56.4 kD and a theoretical pI value of 9.29.

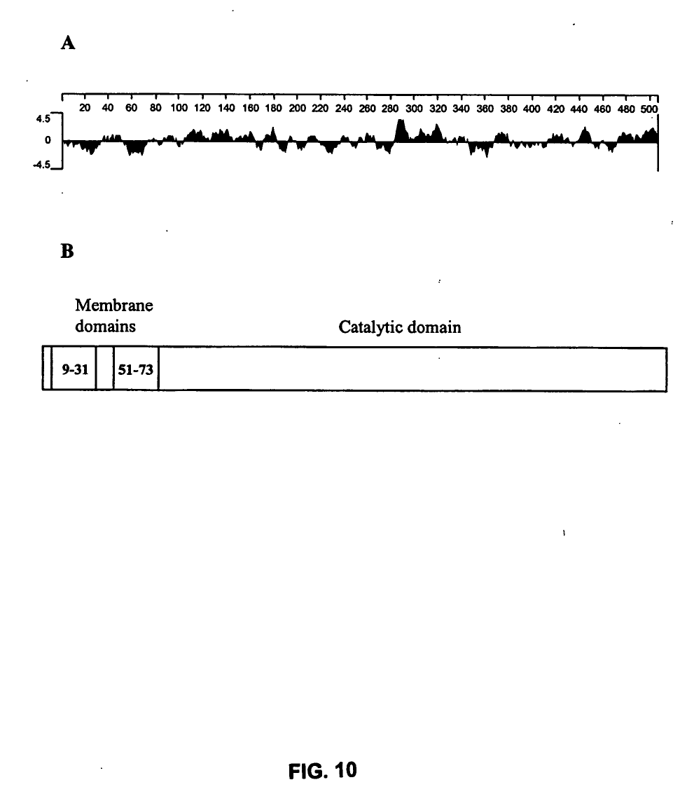

A hydropathy analysis (Kyte-Doolittle) of the amino acid sequence of the Crambe FAE revealed several hydrophobic domains (FIG. 10A). Protein analyses with the TMAP algorithm (Persson and Agros, 1994) predicted two N-terminal transmembrane domains, the first corresponding to amino acid residues 9 to 31 and the second domain spanning residues 51 to 73 (FIG. 10B).

### Example 15

Functional Heterologous Expression of the Crambe abyssinica FAE in Yeast Cells

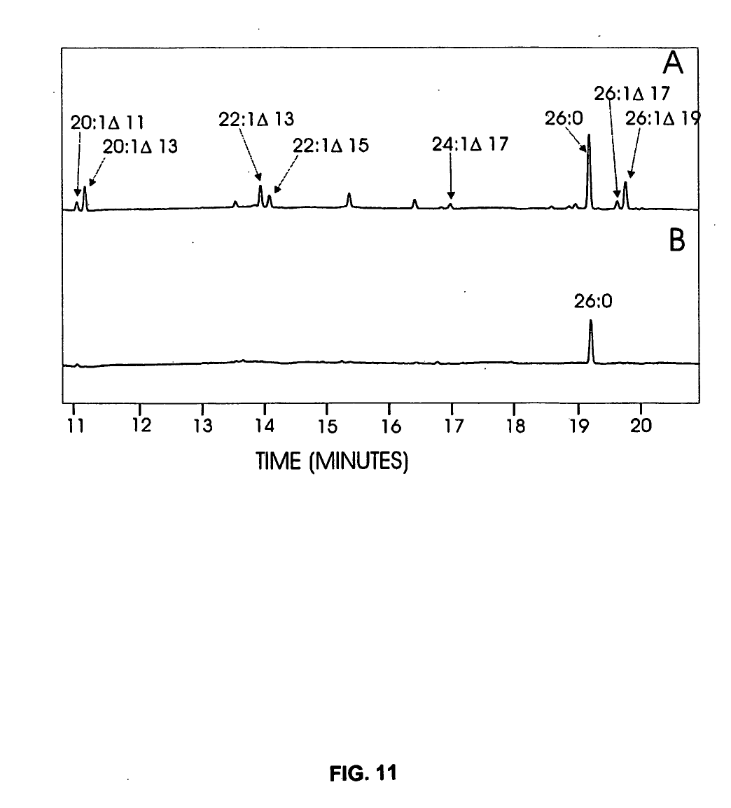

To study the function of the protein encoded by the Crambe FAE, the coding region was linked to the GAL1-inducible promoter in the yeast expression vector pYES2.1/V5-His-TOPO and transformed into S. cerevisiae strain InvSci1 yeast cells. As shown in FIG. 11, yeast cells transformed with the plasmid containing the Crambe FAE open reading frame were found to have an accumulation of 20:1c11, 20:1c13, 22:1c13, 22:1c15 and 26:1c19; these are not present in wild-type yeast cells. In particular, the capability of the Crambe FAE to synthesize 20:1 c11 and 22:1 c13 are directly of interest for our target oil compositional changes as outlined in the Background section.

### Example 16

Heterologous Expression of the Crambe FAE in Arabidopsis and in HEAR Brassicaceae—e.g. B. napus; B. carinata

The coding regions of the Crambe FAE was amplified by polymerase chain reaction with primers: F6-forward: 5′-tatctagaATGACGTCCATTAACGTAAAG-3′(lower case-restriction site for XbaI) (SEQ ID NO:16) and R7-reverse: 5′-atggtaccTTAGGACCGACCGTTTTGG-3′ (lower case shows restriction site for KpnI enzyme) (SEQ ID NO: 17) and subsequently cloned behind the napin promoter in respective sites of pSE vector (Jako et al., 2001).

The final binary vector (napin/crambe FAE) was electroporated into Agrobacterium tumefaciens cells strain GV3101 containing helper plasmid pMP90 (Koncz and Schell, 1986). Plasmid integrity was verified by DNA sequencing following its re-isolation from A. tumefaciens and transformation into E. coli.

The binary vector was used to transform A. thaliana plants by the vacuum infiltration method (Clough and Bent, 1998) and high erucic Brassica napus plants using the methods of DeBlock et al, (1989) and B. carinata plants using the method of Babic et al., (1998).

### Example 17

Heterologous Expression of the Arabidopsis thaliana FAE in HEAR Brassicaceae—e.g. B. napus cv Hero and in B. carinata.

The results from Millar and Kunst (1997) demonstrated that the heterologous expression of the FAE gene alone is sufficient for the production of VLCFAs and that it is the condensing enzyme that determines the quantity and the chain length of VLCFA synthesized by the microsomal fatty acid elongation complex.

Plant Material

High erucic acid Brassica napus L. cultivar Hero (Scarth et al., 1991) was obtained from the Plant Science Department of the University of Manitoba (Winnipeg, Canada). B. napus canola cultivar Westar was obtained courtesy of G. Rakow (Agriculture and Agri-Food Canada Research Center, Saskatoon). All experimental control and transgenic B. napus lines were grown simultaneously in the Kristjanson Biotechnology Complex greenhouses (Saskatoon) under natural light conditions supplemented with high-pressure sodium lamps with a 16 h photoperiod (16 light and 8 h of darkness) at 22° C. and a relative humidity of 25 to 30%.

B. napus cv. Hero SLC1-1 transgenic lines containing a yeast sn-2 lyso-phosphatidic acid acyltransferase (LPAT; EC 2.3.1.51) were produced and characterized as described previously (Zou et al. 1997). PCR and Southern analyses of the transgenic lines selected for further biochemical characterization and field testing showed that all of the lines contained a single SLC1-1 insert.

Lipid Substrates, Chemicals and Biochemicals

[1-¹⁴C] oleic acid (2.15×10⁹ Bq·mmol⁻¹) was purchased from Amersham Canada, Ltd. (Oakville, ON) and [1-¹⁴C]-labeled oleic acid was converted to the corresponding labeled oleoyl-CoA using the method described by Taylor et al. (1990). Specific activity was adjusted as required by diluting with authentic unlabeled standard. Unlabeled oleoyl-CoA, malonyl-CoA, ATP, CoA-SH, NADH, NADPH, sodium acetate and most other biochemicals were purchased from Sigma. The 15:0 and 17:0 standards were supplied by Supelco Canada, Ltd. (Oakville, ON). HPLC-grade solvents (Omni-Solv, BDH Chemicals, Toronto, ON) were used throughout these studies.

FAE1 Transformation Vector

Drs A. Millar and L. Kunst (from the Dept. of Botany, University of British Columbia, Canada) kindly provided the binary vector pNap:FAE1/NGKM (FIG. 1) containing the Arabidopsis thaliana FAE1 coding region under the control of seed-specific, napin promoter. The binary vector was introduced by electroporation into the Agrobacterium tumefaciens strain GV3101 bearing helper plasmid pMP90 and used in transformation experiments.

Transformation of Brassica napus cv Hero with the FAE1 Gene

Cotyledonary-petioles were excised from five to seven-day-old seedlings of the canola cv. Westar, and used as explants in transformation experiments. The transformation was carried out according to the method developed by Moloney et al., (1989).

Hypocotyls were excised from five to seven-day-old seedlings of the HEAR cv. Hero, and cut into 5 to 7 mm segments. The hypocotyl explants were transformed using the method developed by DeBlock et al., (1989).

Experimental wild-type control plants were regenerated in vitro from the cotyledonary petioles and/or hypocotyl explants. Control explants were not co-cultivated with A. tumefaciens. However, with this exception, control explants were subjected to all the other experimental procedures and conditions applied to explants that were co-cultivated with Agrobacterium (and from which transformed shoots were developed).

Control and transformed shoots were rooted in vitro on rooting medium without kanamycin or with 25 μg·mL⁻¹ kanamycin, respectively. Plants with well-developed roots were transferred to soil and grown to maturity. Developing and mature seed from self-pollinated control and transgenic lines grown in the greenhouse, were harvested and subjected to molecular and biochemical analyses.

Molecular Analyses of Transgenic Plants

All molecular analyses (plasmid preparation, polymerase chain reaction (PCR), restriction digestion, DNA gel blot analyses etc.) were performed by methods prescribed by Sambrook et al. (1989) or Ausubel et al. (1995).

PCR Amplification of the Partial Expression Cassette NAP/FAE1/NOS

To check for integration of the napin:FAE1:nos transgene construct into the genome of putative transgenic plants, leaf tissue from T₀ plants was collected and genomic DNA isolated. This DNA was used as a template to amplify the partial expression cassette NAP/FAE1/NOS using oligonucleotide primer NN-3 (5′-TTTCTTCGCCACTTGTCACTCC-3′) (SEQ ID NO:18) which was designed according to the promoter region of the napin gene (position 948-969) and primer NN-4 (5′-CGCGCTATATTTTGTTTTCTA-3′) (SEQ ID NO:19) which was designed according to the nopaline-synthase 3′ UTR sequence (position 1753-1773). The total size of the expected PCR product is ca. 2.0 Kb (0.197 Kb of napin promoter region+1.608 Kb FAE1 coding region+0.204 Kb of nopaline-synthase 3′ UTR region).

Seed Lipid and Protein Analyses

The total fatty acid content and acyl composition of seed lipids was determined by GC of the FAMEs (Fatty Acid Methyl Esters) with either 15:0 or 17:0 free fatty acid added as an internal standard, as described previously (Zou et al, 1997).

For analyses of the FAE1 transgenic progeny, single seeds were cut with a scalpel into small pieces and an internal standard (15:0 free fatty acid) and 1 mL of 3 M methanolic-HCl (Supelco Canada, Ltd.) were added. Transmethylation was performed at 80° C. for 2 h. Reaction mixtures were cooled on ice and 2 mL of 9 g L⁻¹ NaCl was added. The mixture was extracted three times with 2 mL of hexane and then the hexane extracts were combined and taken to dryness under nitrogen. The acyl composition was determined by GC of the FAMEs on a Hewlett-Packard model 5890 gas chromatograph fitted with a DB-23 column (30 m×0.25 mm; film thickness, 0.25 μm; J & W Scientific, Folsom, Calif.). The GC conditions were: injector temperature and flame ionization detector temperature, 250° C.; running temperature program, 180° C. for 1 min, then increasing at 4° C./min to 240° C. and holding this temperature for 10 min. Data from 10 single seed runs of each FAE1 transgenic line were averaged.

For the SLC1-1 and FAE1 field trial progeny, a Near-Infrared Reflectance (NIR) method was used to estimate oil and protein content based on AOCS Procedure Am 1-92 (Firestone, 1998) using the NIR System 6500 (Foss North America), with software packages NEWISI and WINSI (Infrasoft International LLC). The sample size for NIR scanning was about 4.5 g, enough to fill the ring cup. The oil and protein contents as determined by NIR were calibrated against data obtained from NMR and Leco Protein Analyzer/Kjeldahl analyses (performed with a standard set of HEAR seed samples) (Tkachuk, 1981), respectively, and certified by the Canadian Grain Commission.

Elongase Assays of FAE1 Transgenics

Developing seeds were harvested 30 to 35 Days After Pollination (DAP) frozen immediately in liquid nitrogen and stored at −70° C. until homogenized. Seeds (approx. 20) were ground in a cold mortar at 0° C. in 2 mL grinding buffer (100 mM HEPES pH 7.4, 400 mM Sorbitol, 2.5 mM EGTA, 2.5 mM EDTA, 5 mM MgCl₂, 1 mM DTT, PVPP 150 mg·mL⁻¹).

The slurried homogenate was filtered through 1 layer of Miracloth and used to perform elongation assays as described by Taylor et al., (1992). In the standard reaction mixture, 0.2 to 0.5 mg of protein was incubated in shaking water-bath (100 rpm) at 30° C. for 45 min at pH 7.2 with 90 mM Hepes-NaOH, 1 mM ATP, 1 mM CoA-SH, 0.5 mM NADH, 0.5 mM NADPH, 2 mM MgCl₂, 1 mM malonyl-CoA+18 μM [1-¹⁴C] oleoyl-CoA (3.7×10² Bq·nmol⁻¹) in final volume of 500 μL. In each set of reactions, the amount of homogenate protein added was normalized. Reactions were stopped by adding 3 mL of 100 g L⁻ KOH in methanol and the mixtures were heated at 80° C. for 1 h to saponify the acyl lipids and acyl-CoAs. The tubes were cooled on ice and two-2 mL hexane washes were performed to remove non-saponifiable material. These hexane washes were discarded, and 1 mL water was added to the reaction mixtures. The mixture was then acidified by adding 650 μL concentrated 12 M HCl, extracted twice with 2 mL hexane, the hexane extracts combined and dried under N₂. Samples were transmethylated with 3M methanolic-HCl at 80° C. for 1 h. 2 mL of 9 g L⁻¹ NaCl was added, samples were extracted with 2× with 1 mL hexane, dried under N₂, taken up in 110 μL of acetonitrile and quantified by radio-HPLC as described previously (Taylor et al., 1992b).

Field Trials and Analysis of Progeny

All field trials were conducted by the Saskatchewan Wheat Pool at Rosthern, Saskatchewan (Saskatoon farmzone) in the two successive years. The first field trial growing season (26 May-21 September) exhibited 1519 growing degree days, 2309 crop heat units and 172.4 mm of precipitation accumulation. The second field trial growing season (26 May-21 September) exhibited 757 growing degree-days, 1278 crop heat units and 167.5 mm of precipitation accumulation (http://www.farmzone.com/report/climate.asp).

In the first field trial, nineteen SLC1-1 T₃ transgenic lines were field tested in a nursery trial. Transgenics or control lines were planted in a random block design in 3 m rows, with ca 100 seeds per row with 60 cm between rows. Data were collected from 2 to 6 rows of each transgenic line and 18 rows of non-transformed Hero control lines.

In the second field trial, 37 B. napus cv. Hero FAE1 transgenic T₂ lines were field-tested in a nursery trial. Transgenics or control lines were planted in a random block design in 3 m rows, with ca 100 seeds per row with 60 cm between rows. There were two rows of each line.

In the second field trial, seventeen T₄ SLC1-1 transgenic B. napus cv. Hero lines were selected for yield and quality assessment in the field. The SLC1-1 yield field trials were of a random block design. Each plot was ca 6 m² (5 rows wide at 17.8 cm spacing, and 6 m long in size). The T₄ field-grown lines (leaf material) were sampled and analyzed by PCR to confirm the presence of the 0.95 kb SLC1-1 insert, using the primers OM087 (5′-AGAGAGAGGGATCCATGAGTGTGATAGGTAGG-3′) (SEQ ID NO:20) and OM088 (5′-GAGGAAGAAGGATCCGGGTCTATATACTACTCT-3′) (SEQ ID NO:21) which were designed according to the 5′ and 3′ end sequences, respectively, of the SLC1-1 gene as described by Zou et al, (1997).

Analyses were conducted on the progeny (T₅ seed) from triplicate plots. The oil content data collected for each line in this trial were analyzed using the Anova-Fisher's LSD method (P≦0.05) and Tukey's pairwise comparison method in the Minitab Statistical Software Suite Release 12 (Minitab, Inc. State College Pa. 16801-3008).

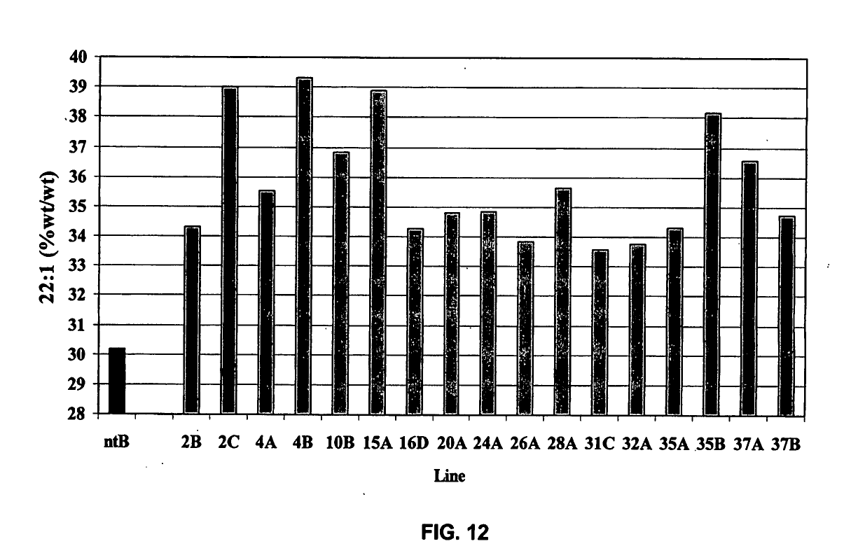

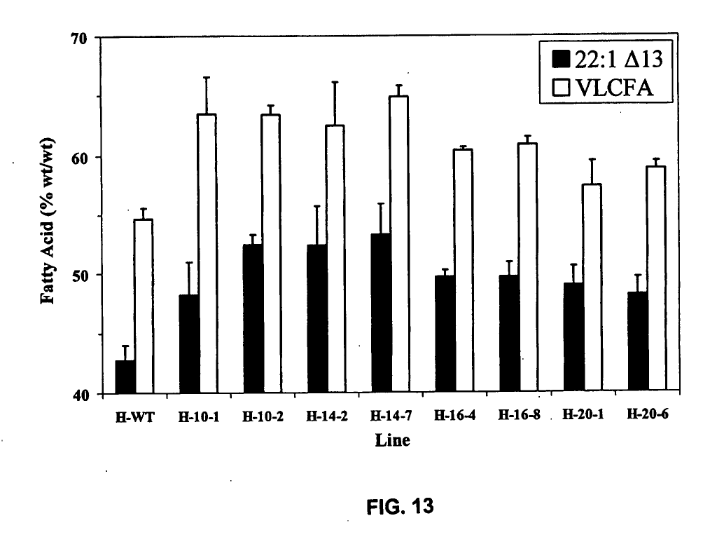

The heterologous expression of the A. thaliana seed specific condensing enzyme FAE1 in our target B. napus HEA cultivar (cv.) Hero resulted in increased levels of eicosenoic, erucic and total VLCFA in our transgenic lines (FIG. 12).

### Example 18

Measure of Elongase Complex Activity in Mid-Developing Seed of B. napus cv Hero Following Heterologous Expression of the Arabidopsis thaliana FAE.

The in vitro assays of elongase activity with homogenates from developing seeds at 30 and 35 DAP (Days After Pollination) from Hero/FAE1 transgenic lines and Hero wild-type controls showed 22 to 100% increase in total elongase activity in transgenic lines when compared to the wild-type controls (Table IV).

### Example 19

Radio-HPLC Measurement of Elongase Complex Activity in Mid-Developing Seed of B. napus cv Hero Following Heterologous Expression of the Arabidopsis thaliana FAE; Shows Arabidopsis thaliana FAE has Preference for Elongating 18:1 to 20:1.

The reverse-phase HPLC (High Pressure Liquid Chromatography) analyses of transgenic lines and wild-type control lines showed that the amounts of both elongation products eicosenoic acid (20:1) and erucic acid (22:1) were higher in transgenic lines with the amounts of 20:1 elongation product being substantially higher in transgenic lines than in the wild-type controls which confirms the functional expression of A. thaliana FAE1 gene in transgenic cv. Hero lines and shows that Arabidopsis condensing enzyme prefers 18:1 over 20:1, as a substrate.

### Example 20

Transgenic Field Trials of B. Napus cv Hero T₃ Generation Following Heterologous Expression of the Arabidopsis thaliana FAE.

The performance of our Hero/FAE1 transgenic lines was tested in the field. Thirty seven T₂ transgenic lines were grown in nursery trials. From the GC (Gas Chromatography) fatty acid methyl ester analyses the erucic acid proportions and oil content of mature T₃ seed from our best transgenic lines showed 8-11% increase in erucic acid proportions and 2-4.8% increase in oil content when compared with the wild type controls (Table V).

### Example 21

Cultivar Development of B. napus cv Hero: Following Heterologous Expression of the Arabidopsis thaliana FAE, the Best Performing Field Trial Lines were Converted to Homozygous Doubled Haploid Lines.

Homozygous lines were produced from selected transgenic lines using microspore-derived embryo technology. The double haploid progeny were analyzed and breeding lines were identified. Seed increases were performed for transgenic field trials and for germplasm development. Using microspore culture technique followed by colchicine treatment doubled haploid lines (DH) were produced from our best Hero/FAE1 transgenic lines. These lines were grown in the greenhouse under the same growth conditions as wild-type Hero control lines. Our best DH transgenic lines showed stable increases in erucic acid proportions with 58-59% erucic acid, while control lines had on average 48% erucic acid in the seed oil. Hero/FAE1 transgenic homozygous DH lines and wild-type control lines can be re-tested in transgenic field trials.

### Example 22

Cultivar Development of B. napus cv Hero: Transgenic Field Trials of Selected DH Lines of B. napus cv Hero Homozygous for the Heterologously Expressed Arabidopsis thaliana FAE.

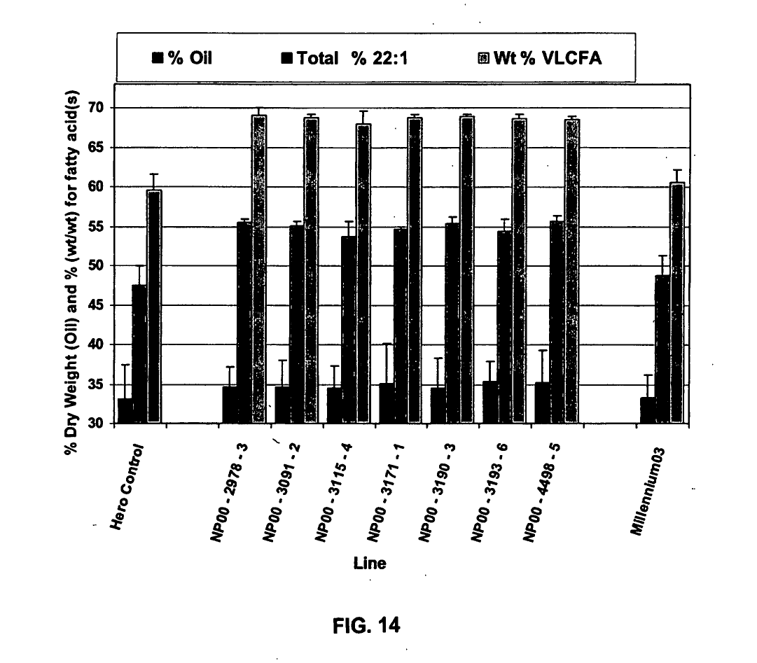

Ten pure double-haploid (DH) B. napus cv. Hero transgenic lines expressing the A. thaliana FAE1 gene were developed in collaboration with the Saskatchewan Wheat Pool. These transgenic lines were tested in the field by the team of breeders from the University of Manitoba (leading breeder—Peter McVetty). Double-haploid (DH) transgenic Hero/FAE1 were subjected to field trials planned and conducted by Peter McVetty at the University of Manitoba. The seed was harvested individually from 5 plants in 3 replicates (15 plants total) for each DH line and GC analyses of seed oil content and oil composition were conducted. The results showed that all DH lines have increased erucic acid content in their seed oil (Table VI) with the 5 best lines having the erucic acid content from 7.5 to 8.2% over that found in the wild-type c.v. Hero field-trial-grown control seed. In addition, our best DH Hero/FAE1 transgenic lines have shown increase of up to 7.0% in erucic acid content when compared to c.v. Millenium field-trial-grown control seed (FIG. 14).

### Example 23

Combining the Effects of FAE Transgenes by Performing Crosses and Breeding Experiments:

Alternatively, individual FAE transgenic lines containing the nasturtium FAE gene, or the Crambe FAE gene, or combinations of these two FAE genes with the A. thaliana FAE (ideally homozygous for the FAE transgene(s), can be selected and then be used for production of pure, dihaploid (DH) lines. These DH lines can then be used for crosses and breeding experiments to produce elite HEAR Brassica cultivars.

TABLE I
Fatty acid composition of transformed tobacco calli.
Results represent the average (±SE) of tén measurements using independent calli.
Constructs: RD = Control (plasmid only) transgenic calli; SF = 35S: T. majus FAE transgenic calli;
SMF = 35S: mutated T. majus FAE transgenic calli.

| | Fatty acid composition (% (wt/wt) of total fatty acids) [% increase]* | | | | | | | |
|---|---|---|---|---|---|---|---|---|
| Construct | 16:0 | 18:0 | 20:0 | 22:0 | 24:0 | 26:0 | LCFA | VLCFA |
| RD | 20.38 ± 0.12 | 7.99 ± 0.26 | 1.32 ± 0.03 | 0.59 ± 0.03 | 0.70 ± 0.03 | 0.89 ± 0.16 | 96.28 ± 0.31 | 3.72 ± 0.31 |
| SF | 18.01 ± 0.42 | 5.23 ± 0.41 | 1.58 ± 0.54 | 1.32 ± 0.16 | 1.93 ± 0.27 | 1.31 ± 0.20 | 91.37 ± 0.84 | 8.63 ± 0.84 |
| | | | [19.7] | [123.9] | [175.7] | [147.2] | (0.84) | [131.9] |
| SMF | 19.48 ± 0.34 | 7.12 ± 0.19 | 1.30 ± 0.02 | 0.57 ± 0.03 | 0.73 ± 0.04 | 1.01 ± 0.32 | 95.59 ± 0.40 | 4.41 ± 0.40 |
| | | | | | | | | [18.5] |

*relative to value for calli from RD: the tobacco control (plasmid only) calli, set at 100%.

TABLE II
Fatty acid composition of transformed tobacco leaves.
Results represent the average (±SE) of ten measurements using leaf discs from ten independent transgenic
plants. Constructs: RD = Control (plasmid only) transgenic leaves; SF = 35S: T. majus FAE transgenic leaves;
SMF = 35S: mutated T. majus FAE transgenic leaves.

| | Fatty acid composition (% (wt/wt) of total fatty acids) [% increase]* | | | | | | | | |
|---|---|---|---|---|---|---|---|---|---|
| Construct | 16:0 | 18:0 | 18:3 | 20:0 | 20:1c11 | 22:0 | 24:0 | LCFA | VLCFA |
| RD | 16.32 ± 0.14 | 3.94 ± 0.11 | 53.30 ± 0.72 | 0.53 ± 0.02 | 1.18 ± 0.00 | 0.27 ± 0.01 | 2.74 ± 0.09 | 93.77 ± 0.29 | 6.23 ± 0.29 |
| SF | 15.83 ± 0.14 | 3.35 ± 0.12 | 47.02 ± 0.66 | 0.91 ± 0.12 | 2.34 ± 0.12 | 0.42 ± 0.02 | 4.14 ± 0.15 | 88.64 ± 0.35 | 11.36 ± 0.35 |
| | | | | [71.7] | [98.3] | [55.6] | [51.1] | | [82.3] |
| SMF | 15.53 ± 0.17 | 4.00 ± 0.12 | 47.25 ± 0.85 | 0.98 ± 0.16 | 2.61 ± 0.02 | 0.30 ± 0.01 | 3.24 ± 0.08 | 90.05 ± 0.28 | 9.95 ± 0.28 |
| | | | | [84.5] | [121.2] | | [18.2] | | [59.7] |

*relative to value for leaves from RD: the tobacco control (plasmid only) plants, set at 100%.

TABLE III
Fatty acid composition of transgenic Arabidopsis T₂ seeds.
Results represent the average ± SE of triplicate measurements using 200 seeds from 25 independent Arabidopsis transgenic
lines. Constructs: RD = Control (plasmid only) transgenic seeds; NF = Napin: T. majus FAE transgenic seeds.

| | Fatty acid composition (% (wt/wt) of total fatty acids) {Range} [% increase]* | | | | | | | |
|---|---|---|---|---|---|---|---|---|
| Construct | 18:0 | 20:1c11 | 22:0 | 22:1c13 | 24:0 | 24:1c15 | LCFA | VLCFA |
| RD | 3.72 ± 0.07 | 19.87 ± 0.26 | 0.30 ± 0.01 | 2.12 ± 0.05 | 0.11 ± 0.01 | 0.19 ± 0.01 | 70.15 ± 0.22 | 29.85 ± 0.22 |
| | {3.35-4.03} | {17.97-20.86} | {0.27-0.34} | {1.88-2.28} | {0.09-0.15} | {0.15-0.24} | {69.35-71.36} | {28.64-30.65} |
| NF | 2.57 ± 0.10 | 12.78 ± 0.42 | 1.57 ± 0.12 | 9.63 ± 0.59 | 0.46 ± 0.03 | 0.46 ± 0.03 | 68.77 ± 0.47 | 31.30 ± 0.47 |
| | {1.58-3.31} | {8.87-16.85} | {0.66-2.78} | {4.43-15.57} | {0.24-0.65} | {0.29-0.70} | {65.53-72.06} | {27.94-34.47} |
| | | | [423.3] | [354.2] | [318.2] | [142.1] | | [4.8] |

*relative to value for seeds from RD: the Arabidopsis control (plasmid only) plants, set at 100%.

TABLE IV
Elongase activity in homogenates from developing seed
of B. napus cv. Hero non-transformed wild-type (H-WT)
lines and Hero/FAE1 T₂ transgenic lines H-10-2
(Assay set 1); H-20-1 (Assay set 2); T₃ transgenic line
H-14-7-5 (Assay set 3). Data are the means ± S.D. from assays
of 2 to 5 samples. Homogenates were incubated at 30° C. in a
water bath with shaking at 100 rpm for 45 min with 18 μM
[1−¹⁴C.] 18:1-CoA (3.7 × 10² Bq nmol⁻¹) and
1 mM malonyl-CoA in the presence of 1 mM CoA-SH, 1 mM
ATP, 0.5 mM NADH, 0.5 mM NADPH and 2 mM MgCl₂.
After incubation, reaction mixtures were saponified,
transmethylated and analyzed by HPLC equipped with a
flow through scintillation counter (radio-HPLC).

| | | | | Total |
|---|---|---|---|---|
| | | | | 20:1 Δ11 ± |
| | | 20:1 Δ11 | 22:1 Δ13 | 22:1 Δ13 |
| | | pmol/min/mg protein | | |
| zebra | Line | | | [% increase]^{a} |
| Set 1 | H-WT | 493 ± 35 | 60 ± 3 | 553 ± 38 |
| | H-10-2 | 978 ± 43 | 121 ± 7 | 1099 ± 50 |
| | | | | [99] |
| Set 2 | H-WT | 408 ± 48 | 139 ± 14 | 547 ± 62 |
| | H-20-1 | 462 ± 29 | 203 ± 1 | 665 ± 30 |
| | | | | [22] |
| Set 3 | H-WT | 298 ± 22 | 21 ± 3 | 319 ± 25 |
| | H-14-7-5 | 462 ± 6 | 12 ± 4 | 474 ± 10 |
| | | | | [49] |

TABLE V
The proportions of erucic acid, total
VLCFAs and oil content in seed of non-transformed,
B. napus cv. Hero wild-type controls (H-WT) and T₃
seed of five selected Hero/FAE1 transgenic
lines (H-10-2 to H-14-7) from field trials.

| | | |
|---|---|---|
| | % Total VLCFAs | Oil Content |
| % 22:1 (w/w) | (w/w) | (% of DW) |

| | |
|---|---|
| Line | [% increase]^{a} |

| | | | |
|---|---|---|---|
| H-WT | 48.1 | 57.6 | 44.2 |
| H-10-2 | 59.1 [22.9] | 67.4 [17.0] | 47.2 [6.8] |
| H-10-5 | 56.5 [17.5] | 64.7 [12.3] | 46.2 [4.5] |
| H-10-6 | 56.7 [17.9] | 64.8 [12.5] | 46.5 [5.2] |
| H-10-10 | 57.1 [18.7] | 65.2 [13.1] | 44.7 [1.1] |
| H-14-7 | 56.2 [16.8] | 63.9 [10.9] | 49.0 [10.9] |

^{a}Relative to non-transformed wild type Hero control.

TABLE VI
Proportions of erucic acid and total very long chain fatty acids
(VLCFA in DH B. napus c.v. Hero/FAE1 transgenic lines and c.v. Hero
and c.v. Millennium wild-type control plants from transgenic field
trials. The results represent average ± SD of twelve seed samples
from ten plants for each transgenic DH line and wild-type (WT) controls.
Millenium is an elite commercially grown cv.

| | 22:1 Proportions | VLCFA Proportions |
|---|---|---|
| Line | (% w/w) | (% w/w) |
| Hero WT control | 47.50 ± 2.47 | 59.64 ± 1.93 |
| NP00-2978-3 | 55.46 ± 0.52 [8.0]* | 69.05 ± 0.98 [9.4] |
| NP00-3030-3 | 51.80 ± 1.67 [4.3] | 67.42 ± 0.89 [7.8] |
| NP00-3091-2 | 55.03 ± 0.63 [7.5] | 68.77 ± 0.40 [9.1] |
| NP00-3094-5 | 54.87 ± 1.10 [7.4] | 68.79 ± 0.49 [9.1] |
| NP00-3098-2 | 55.43 ± 0.26 [7.9] | 68.98 ± 0.33 [9.3] |
| NP00-3115-4 | 53.67 ± 1.96 [6.2] | 67.98 ± 1.59 [8.3] |
| NP00-3171-1 | 54.65 ± 0.32 [7.1] | 68.81 ± 0.33 [9.2] |
| NP00-3190-3 | 55.35 ± 0.91 [7.8] | 68.86 ± 0.35 [9.2] |
| NP00-3193-6 | 54.36 ± 1.59 [6.9] | 68.65 ± 0.51 [9.0] |
| NP00-4498-5 | 55.69 ± 0.63 [8.2] | 68.56 ± 0.30 [8.9] |
| Milennium WT control | 48.73 ± 2.55 | 60.57 ± 1.55 |

*[% increase] relative to non-transformed wild type Hero control

### LITERATURE CITED

Ausubel, F. M., Brent, R., Kingston, R. E., Moore, D. D., Seidman, J. G., Smith, J. A., and Stuhl, K. (1995). Current Protocols in Molecular Biology. (Wiley, New York). Vols. 1, 2, & 3.
Babic V., Datla R. S., Scoles G. J. and Keller W. A (1998) Development of an efficient Agrobacterium-mediated transformation system for Brassica carinata. Plant Cell Reports 17: 183-188.
Bjellqvist B, Hughes G J, Pasquali C H, Paquet N, Ravier F, Sanchez J-C. h, Frutiger S, Hochstrasser D. (1993) The focusing positions of polypeptides in immobilized pH gradients can be predicted from their amino acid sequences. Electrophoresis 14: 1023-1031.
Bjellqvist B, Basse B, Olsen E, Celis J E (1994) Reference points for comparisons of two-dimensional maps of proteins from different human cell types defined in a pH scale where isoelectric points correlate with polypeptide compositions. Electrophoresis 15:529-539.
Bradford M M (1976) A rapid and sensitive method for the quantification of microgram quantities of protein utilizing the principle of protein-dye binding. Anal Biochem 72: 248-254.
Chen J, Greenblatt I M, Dellaporta S. L (1992) Molecular analysis of Ac transposition and DNA replication. Genetics 130: 665-676.
Clough S J, Bent A F (1998) Floral dip: a simplified method for Agrobacterium-mediated transformation of Arabidopsis thaliana Plant J 16: 735-743.
Datla, R., Hammerlindl, J. K., Panchuk, B., Pelcher, L. E., and Keller, W. A. (1992). Modified binary plant transformation vectors with the wild-type gene encoding NPTII. Gene 122, 383-384.
DeBlock, M, D. DeBrouwer, and P. Tenning. 1989. Transformation of Brassica napus and Brassica oleracea using Agrobacterium tumefaciens and the expression of the bar and neo genes in the transgenic plants. Plant Physiol. 91:694-701.
Jako C, Kumar A, Wei Y, Zou J-T, Barton D L, Giblin E M, Covello P S, Taylor D C (2001) Seed-specific over-expression of an Arabidopsis thaliana cDNA encoding a diacylglycerol acyltransferase enhances seed oil content and seed weight. Plant Physiol 126: 861-874.
Jako C, Coutu C, Roewer I, Reed D W, Pelcher L E, Covello P S (2002) Probing carotenoid biosynthesis in developing seed coats of Bixa oreallana (Bixaceae) through expressed sequence tag analysis. Plant Sci 163: 141-145.
Katavic V, Friesen W, Barton D L, Gossen K K, Giblin E M, Luciw T, An J, Zou J, MacKenzie S L, Keller W A, Males D and Taylor D C (2000) Utility of the Arabidopsis FAE1 and Yeast SLC1-1 Genes for improvements in Erucic Acid and Oil Content in Rapeseed. Biochem. Soc. Trans. 28(6): 935-937.
Katavic V, Friesen W, Barton D L, Gossen K K, Giblin E M, Luciw T, An J, Zou J-T, MacKenzie S L, Keller W A, Males D, Taylor D C (2001) Improving erucic acid content in rapeseed through biotechnology: What can the Arabidopsis FAE1 and the yeast SLC1-1 genes contribute? Crop Sci 41: 739-747.
Lindstrom J T, Vodkin L O (1991) A soybean cell wall protein is affected by seed color genotype. Plant Cell 3: 561-571.
Mietkiewska E., Giblin E M., Wang S., Barton D L., Dirpaul J., Brost J M., Katavic V., Taylor D C. (2004) Seed-specific heterologous expression of a nasturtium FAE gene in Arabidopsis results in a dramatic increase in the proportion of erucic acid. Plant Physiology 136: 2665-2675.
Millar A J, Kunst L (1997) Very long chain fatty acid biosynthesis is controlled through the expression and specificity of the condensing enzyme. Plant J 12:121-131.
Moloney, M. M., Walker, J. M., and Sharma, K.K. (1989). High efficiency transformation of Brassica napus using Agrobacterium vectors. Plant Cell Rep. 8, 238-242.
Persson B, Argos P (1994) prediction of transmembrane segments in proteins utilizing multiple sequence alignments. J Mol Biol 237: 182-192.
Taylor D C, Weber N, Hogge L R, Underhill E W (1990) A simple enzymatic method for the preparation of radiolabeled erucoyl-CoA and other long-chain fatty acyl-CoAs and their characterization by mass spectrometry. Analyt Biochem 184: 311-316.
Taylor D C, Magus J R, Bhella J, Zou J-T, Mackenzie S L, Giblin E M, Pass E W, Crosby W L (1992a) Biosynthesis of triacylglycerols in Brassica napus L. cv. Reston; Target: Triercin. In: S L MacKenzie and D C Taylor, eds, Seed Oils for the Future. American Oil Chemists' Society, Champaign, Ill., pp 77-102.
Taylor D C, Barton D L, Rioux K P, Reed D W, Underhill E W, MacKenzie S L, Pomeroy M K, Weber N (1992b) Biosynthesis of acyl lipids containing very-long chain fatty acids in microspore-derived and zygotic embryos of Brassica napus L. cv. Reston. Plant Physiol 99: 1609-1618.

Taylor D C, Katavic V, Zou J-T, MacKenzie S L, Keller W A, An J, Friesen W, Barton D L, Gossen, K K, Giblin E M, Ge Y, Dauk M, Luciw T, Males D (2001) Field-testing of transgenic rapeseed cv. Hero transformed with a yeast sn-2 acyltransferase results in increased oil content, erucic acid content and seed yield. Mol Breeding 8: 317-322.
Zou J-T, Katavic V, Giblin E M, Barton D L, MacKenzie S L, Keller W A, Hu X, Taylor D C (1997) Modification of Seed Oil Content and Acyl Composition in Brassicaceae by Expression of a Yeast sn-2 Acyltransferase Gene. Plant Cell 9: 909-923.

1. An expression vector for transforming a cell, said expression vector comprising a gene coding for a plant fatty acid elongase in reading frame alignment with a promoter capable of increasing expression of said gene, when said transformed cell is in a seed, sufficient to increase the proportion of very long chain monounsaturated fatty acid when compared with a control cell.

2. The expression vector of claim 1 wherein said gene coding for a plant fatty acid elongase is a Nasturtium fatty acid elongase gene.

3. The expression vector of claim 1 wherein said gene coding for a plant fatty acid elongase is a Crambe fatty acid elongase gene.

4. The expression vector of claim 1 wherein said gene coding for a plant fatty acid elongase is an Arabidopsis fatty acid elongase gene.

5. A cell comprising a heterologous gene coding for a heterologous plant fatty acid elongase or allelic variant thereof, said cell being capable of producing an increase in proportion of a very long chain monounsaturated fatty acid when compared a control cell lacking said heterologous gene.

6. The cell of claim 5 wherein said cell is a fungal cell.

7. The cell of claim 6 wherein said fungal cell is a yeast cell.

8. The cell of claim 5 wherein said cell is a plant cell.

9. The cell of claim 5 wherein said cell is a plant seed cell.

10. The plant cell of claim 8 additionally comprising a further heterologous gene coding for an additional heterologous plant fatty acid elongase or allelic variant thereof or a heterologous plant desaturase gene or allelic variant thereof.

11. The plant cell of claim 10 capable of producing oil with an increased content of erucic acid or other very long chain fatty acid (C₂₀ or greater).

12. A plant cell according to claim 8 wherein said heterologous gene codes for a 3-ketoacyl-CoA synthase.

13. A plant cell according to claim 8 wherein said very long chain monounsaturated fatty acid comprises erucic acid.

14. A seed comprising a plurality of plant cells according to claim 8.

15. A plant comprising a plurality of plant cells according to claim 8.

16. A plant according to claim 15 wherein said plant is a dicotyledon.

17. A plant according to claim 15 wherein said plant is a member of the Brassicaceae.

18. A plant according to claim 15 wherein said plant is a member of the Limnanthaceae or Tropaeolaceae or Simmondsia.

19. A plant according to claim 15 wherein said plant is flax (Linum usitatissimu L.).

20. A plant according to claim 17 wherein said plant is of the Brassica genus.

21. A method for altering erucic acid content of a plant-derived oil which method comprises cultivating a plant according to claim 15 and then extracting a plant-derived oil therefrom which oil has altered erucic acid content.

22. Use of a heterologous plant fatty acid elongase gene for altering erucic acid content in a plant.

23. Use of a heterologous plant fatty acid elongase gene for altering the very long chain fatty acid content (C₂₀ or greater) in a plant.
